(19) United States
(12) Patent Application Publication
(10) Pub. No.: US 2021/0324427 A1
(43) Pub. Date: Oct. 21, 2021

(54) GENETICALLY MODIFIED BACTERIUM FOR PRODUCING LACTATE FROM CO2

(71) Applicant: ENOBRAQ, Ramonville-Saint-Agne (FR)

(72) Inventors: Cédric BOISART, Belberaud (FR); Nicolas CHABOT, Ramonvillle-Saint-Agne (FR)

(21) Appl. No.: 16/767,271

(22) PCT Filed: Nov. 30, 2018
(86) PCT No.: PCT/EP2018/083097

§ 371 Date: May 27, 2020

(30) Foreign Application Priority Data
Nov. 30, 2017 (FR) 1761481

(51) Int. Cl. C12P 7/56; C12N 9/04

(52) U.S. Cl. CPC C12P 7/56; C12Y 101/01028; C12Y 101/01027; C12N 9/0006

(57) ABSTRACT

The invention relates to a naturally hydrogen-oxidizing bacterium which is genetically modified to produce lactate from CO₂, said bacterium being genetically modified to overexpress at least one gene encoding a lactate dehydrogenase, and to a process for producing lactate from CO₂ using such a bacterium.

This application hereby incorporates by reference the material of the electronic Sequence Listing filed concurrently herewith. The material in the electronic Sequence Listing is submitted as a text (.txt) file entitled “102335-023 Sequence Listing.txt” created on Nov. 19, 2020, which has a file size of 55 KB, and is herein incorporated by reference in its entirety.

### FIELD OF THE INVENTION

The invention relates to a naturally hydrogen-oxidizing bacterium which is genetically modified to produce lactate from CO₂. The invention also relates to a process for producing lactate, or lactic acid, from CO₂ using such a genetically modified bacterium.

### BACKGROUND OF THE INVENTION

Lactic acid has applications in many industries. For example, lactic acid is used as a precursor of polylactic acid (PLA) which is a fully biodegradable polymer used for example in food packaging. Lactic acid can also be used as an additive, as an antioxidant, acidifier or flavor enhancer by the food industry. In cosmetics, lactic acid is generally used as a bacteriostatic or peeling agent.

At present, the industrial-scale production of lactic acid is mainly based on the fermentation of carbohydrates, in particular glucose, lactose, sucrose or maltose. While many bacteria can cause the transformation of these sugars into lactic acid, only lactic acid bacteria can produce lactic acid exclusively, the other bacteria generally producing a mixture of several organic acids.

In addition, the production of lactic acid from fermentable sugars such as glucose or lactose raises the question of competition between food and production of commodities such as lactic acid.

At the same time, emissions of carbon dioxide (CO₂) into the atmosphere are increasing. Biological systems that fix carbon through natural biochemical metabolic processes, such as algae, have already been used to produce molecules of interest through photosynthetic reactions. However, production yields remain insufficient and limit the economic interest of these systems.

Similarly, methods using genetically modified bacterial cells have been developed to transform sugars into molecules of interest in heterotrophic fermentation systems. Angermayr et al., (2012) describes the overexpression of Bacillus subtilis lactate dehydrogenase in the cyanobacterium Synechocystis. WO 2014/205146 and WO 2015/155790 disclose methanotrophic bacteria whose energy source comes from their carbon substrate. Such systems have several disadvantages. Indeed, heterotrophic fermentation systems are sensitive to contamination, which has a significant impact on production yields. Moreover, these heterotrophic systems do not solve the problems of competition with food, since fermentable sugars are still needed, nor of negative environmental impacts.

There is therefore still a need for microbiological processes to enable the production of molecules, such as lactic acid, in large quantities from CO₂ as the only source of carbon, so as not to compete with food while reducing greenhouse gas emissions.

### SUMMARY OF THE INVENTION

Working on this issue, the inventors have discovered that it is possible to force bacteria that naturally oxidize hydrogen, and capable of producing organic matter from CO₂, into a lactate synthesis pathway, possibly to the detriment of other synthesis pathways. In particular, the inventors have discovered that it is possible to favor an endogenous lactate synthesis pathway, which is not or slightly expressed naturally in the bacterium, by playing on the expression of the gene(s) associated with this synthesis pathway. Thus, the inventors have discovered that hydrogen-oxidizing bacteria can be genetically modified so as to overexpress an endogenous lactate dehydrogenase or a heterologous one, and/or so as to repress the expression of genes involved in a synthesis pathway of molecules competing with the production of lactate, in order to produce lactate from CO₂. More particularly, the inventors have discovered that the bacterium Cupriavidus necator (also called Hydrogenomonas eutrophus, Alcaligenes eutropha, Ralstonia eutropha, or Wautersia eutropha) can be genetically modified so as to overexpress an endogenous lactate dehydrogenase, by acting on the promoter and/or the copy number of the gene encoding for this lactate dehydrogenase in particular, so as to produce lactate from CO₂. Alternatively or complementarily, the production of lactate can be improved by introducing one or more genes expressing a heterologous lactate dehydrogenase and/or by repressing the expression of genes involved in a synthesis pathway of molecules competing with the production of lactate.

The invention therefore relates to a naturally hydrogen-oxidizing bacterium which is genetically modified to produce lactate from CO₂, said bacterium being genetically modified to overexpress at least one gene encoding a lactate dehydrogenase.

According to the invention, the bacterium can be genetically modified to overexpress an endogenous and/or exogenous lactate dehydrogenase.

The invention also relates to the use of a bacterium according to the invention for producing lactate from CO₂, preferentially for producing exclusively L-lactate, or for producing exclusively D-lactate.

The invention also relates to a process for producing lactate from CO₂, comprising the steps consisting in
culturing the bacterium with CO₂ as its sole carbon source, then
recovering the lactate.

### DESCRIPTION OF THE FIGURES

FIG. 1 shows a naturally hydrogen-oxidizing bacterium according to the invention capable of producing lactate from CO₂ and H₂.

FIG. 2 shows the different metabolic pathways naturally present in Cupriavidus necator, including glycolysis and the Calvin cycle.

FIG. 3 shows the different genetic modifications (overexpression and/or inhibition of gene expression) that can be carried out in Cupriavidus necator to promote the production of L-lactate from CO₂.

FIG. 4 shows the different genetic modifications (overexpression and/or inhibition of gene expression) that can be performed in Cupriavidus necator to promote the production of D-lactate from CO₂.

FIG. 5 is a table summarizing the various abbreviations used in the description and in FIGS. 2, 3 and 4.

FIG. 6 shows the production of lactate by the bacterium CN0002 from fructose.

FIG. 7 shows the production of lactate by the naturally hydrogen-oxidizing bacterium CN0002 and genetically modified to produce lactate from CO₂.

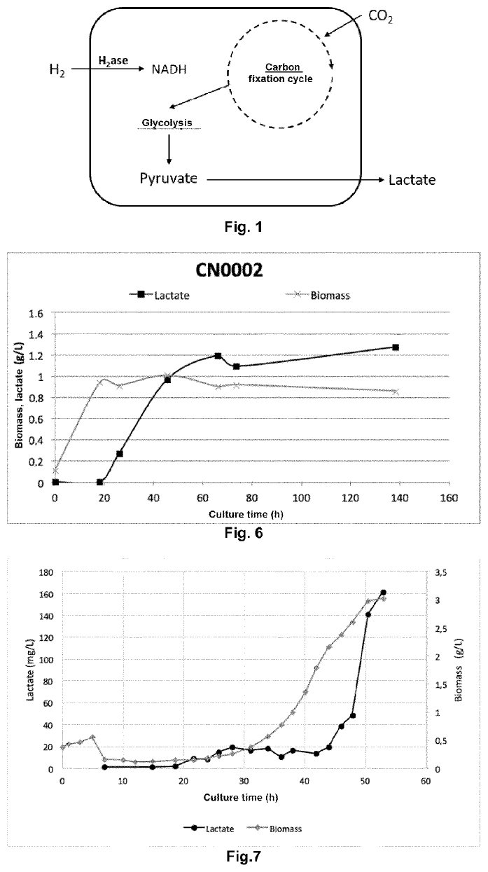

### DETAILED DESCRIPTION OF THE INVENTION

The invention relates to a naturally hydrogen-oxidizing bacterium which is genetically modified to produce lactate from CO₂, said bacterium being genetically modified to overexpress at least one gene encoding for a lactate dehydrogenase.

In the context of the invention, a “naturally hydrogen-oxidizing” bacterium means a bacterium capable, without prior genetic manipulation, of using hydrogen gas as an electron donor and oxygen as an electron acceptor, and capable of binding carbon dioxide. These bacteria are also called “Knallgas” bacteria. Naturally hydrogen-oxidizing bacteria require carbon dioxide as a source of carbon and hydrogen as a source of energy.

According to the invention, the bacterium naturally oxidizing hydrogen is preferentially selected from Ralstonia sp., Cupriavidus sp., Hydrogenobacter sp., Rhodococcus sp., Hydrogenovibrio sp.; Rhodopseudomonas sp., Rhodobacter sp, Aquifex sp., Cupriavidus sp., Couynebacterium sp., Nocardia sp., Rhodopseudomonas sp., Rhodospirillum sp., Rhodococcus sp., Rhizobium sp., Thiocapsa sp., Pseudomonas sp., Hydrogenomonas sp., Hydrogenobacter sp., Hydrogenophilus sp., Hydrogenautresmus sp., Helicobacter sp., Xanthobacter sp., Hydrogenophaga sp., Bradyrhizobium sp., Alcaligenes sp., Amycolata sp., Aquaspirillum sp., Arthrobacter sp., Azospirillum sp., Variovouax sp., Acidovouax sp., Bacillus sp., Calderobacterium sp., Derxia sp., Flavobacterium sp., Microcyclus sp., Mycobacterium sp., Paracoccus sp., Persephonella sp., Renobacter sp., Thermocrinis sp., Wautersia sp., and cyanobacteria such as Anabaena sp.,

In particular, the bacterium is selected from Rhodococcus opacus, Rhodococcus Hydrogenovibrio marinus, Rhodopseudomonas capsulate, Rhodopseudomonas palustris, Rhodobacter sphaeroides, Aquifex pyrophilus, Aquifex aeolicus, Cupriavidus necator, Cupriavidus metallidurans, Couynebacterium autotrophicum, Nocardia autotrophica, Nocardia opaca, Rhodopseudomonas palustris, Rhodopseudomonas capsulate, Rhodopseudomonas viridis, Rhodopseudomonas sulfoviridis, Rhodopseudomonas blastica, Rhodopseudomonas spheroides, Rhodopseudomonas acidophila, Rhodospirillum rubrum, Rhodococcus opacus, Rhizobium japonicum, Thiocapsa roseopersicina, Pseudomonas facilis, Pseudomonas flava, Pseudomonas putida, Pseudomonas hydrogenovoua, Pseudomonas palleronii, Pseudomonas pseudoflava, Pseudomonas saccharophila, Pseudomonas thermophila, Pseudomonas hydrogenothermophila, Hydrogenomonas pantotropha, Hydrogenomonas eutropha, Hydrogenomonas facilis, Hydrogenobacter thermophilus, Hydrogenobacter halophilus, Hydrogenobacter hydrogenophilus, Hydrogenophilus isleticus, Hydrogenophilus thermoluteolus, Hydrogenautresmus marinus, Helicobacter pyloui, Xanthobacter autotrophicus, Xanthobacter flavus, Hydrogenophaga flava, Hydrogenophaga palleronii, Hydrogenophaga pseudoflava, Bradyrhizobium japonicum, Ralstonia eutropha, Alcaligenes eutrophus, Alcaligenes facilis, Alcaligenes hydrogenophilus, Alcaligenes latus, Alcaligenes paradoxus, Alcaligenes ruhletii, Amycolata sp., Aquaspirillum autotrophicum, Arthrobacter strain 11/X, Azospirillum lipoferum, Variovouax paradoxus, Acidovouax facilis, Bacillus schlegelii, Bacillus tusciae, Calderobacterium hydrogenophilum, Derxia gummosa, Flavobacterium autautresmophilum, Microcyclus aquaticus, Mycobacterium goudoniae, Paracoccus denitrificans, Persephonella marina, Persephonella guaymasensis, Renobacter vacuolatum, Thermocrinis ruber, Wautersia sp., Anabaena oscillarioides, Anabaena spiroides and Anabaena cylindrica.

The terms “recombinant bacterium” and “genetically modified bacterium” are used interchangeably herein and refer to bacteria which have been genetically modified to express or overexpress endogenous nucleotide sequences, to express heterologous (exogenous) nucleotide sequences, or which have an alteration of the expression of an endogenous gene.

“Alteration” means that the expression of the gene, or level of a RNA molecule or equivalent RNA molecules encoding one or more polypeptides or polypeptide subunits, or the activity of one or more polypeptides or polypeptide subunits is regulated such that the expression, level or activity is higher or lower than that observed in the absence of the alteration. It is understood that the terms “recombinant bacterium” and “genetically modified bacterium” refer not only to the particular recombinant bacterium but also to the progeny or potential progeny of such a bacterium. As some changes may occur in subsequent generations, due to mutation or environmental influences, this progeny may not be identical to the parent cell, but is still included within the scope of the term as used here.

According to the invention, “overexpression of a gene” means the fact that said gene is more expressed in the bacterium in question than in a bacterium not genetically modified to overexpress said gene, leading to a production or a greater production of the corresponding protein (and more particularly lactate dehydrogenase) and in particular to an increase of more than 20%, more preferentially 30%, 40%, 50%, 60%, 70%, 80%, 90%. According to the invention, such overexpression means the expression of an endogenous lactate dehydrogenase, which is not or slightly expressed in the non-genetically modified bacterium, or the expression of an exogenous lactate dehydrogenase.

In a particular embodiment, the bacterium is selected from naturally hydrogen-oxidizing bacteria having an endogenous lactate dehydrogenase.

Advantageously, the bacterium is selected from Cupriavidus sp., such as Cupriavidus necator, Hydrogenobacter sp., such as Hydrogenobacter thermophilus, Rhodococcus sp., such as Rhodococcus opacus, and Pseudomonas sp., such as Pseudomonas hydrogenothermophila.

The terms “endogenous” or “native” refer to a gene that is normally or naturally present in the genome of the bacterium in question. Conversely, the terms “exogenous” or “heterologous” as used herein in reference to a gene (nucleotide sequence) refer to a gene which is not normally or naturally present in the genome of the bacterium under consideration.

In the case of a bacterium having an endogenous lactate dehydrogenase, it is possible to genetically modify said bacterium so as to allow overexpression of said gene. In particular, it is possible to modify the bacterium so that at least one gene encoding for an endogenous lactate dehydrogenase is under the control of a promoter permitting such overexpression. A promoter refers to the sequence at the 5′ end of the structural gene under consideration and which directs the initiation of transcription. Generally speaking, according to the invention, the usable promoters include constitutive promoters, namely promoters which are active in most cell states and environmental conditions, as well as inducible promoters which are activated or repressed by exogenous physical or chemical stimuli, and which therefore induce a variable level of expression depending on the presence or absence of these stimuli. Alternatively or complementarily, it is possible to genetically modify the endogenous promoter, for example to remove potential inhibitions of its induction. It is also possible to overexpress an endogenous lactate dehydrogenase by multiplying the number of copies of the gene in the genome of the bacterium, by means of plasmids and/or by introducing nucleotide sequences at other loci on the chromosome(s) of the bacterium, etc.

In an embodiment, the bacterium according to the invention is genetically modified to overexpress an endogenous lactate dehydrogenase. Advantageously, the bacterium is genetically modified to overexpress only endogenous L-lactate dehydrogenase or only endogenous D-lactate dehydrogenase.

Tables 1 and 2 below list, as examples, sequences encoding for L-lactate dehydrogenase and D-lactate dehydrogenase, respectively, from different bacteria and the corresponding protein sequences. According to the invention, these sequences can be the target of genetic modifications, including multiplication, to lead to a genetically modified bacterium capable of producing lactate from CO₂.

TABLE 1
Examples of L-lactate dehydrogenas (EC 1.1.1.27)

| Microorganism | Gene | GenBank | Protein sequence |
|---|---|---|---|
| Cupriavidus necator H16 | Idh | CAJ91814.1 | SEQ ID NO: 1 |
| Pediococcus acidilactici | IdhA | 1082254718 | SEQ ID NO: 2 |
| Streptococcus equinus | Idh | KFN85486.1 | SEQ ID NO: 3 |
| (Streptococcus bovis) | | | |
| Bacillus coagulans | Idh | AGU00860.1 | SEQ ID NO: 4 |
| Lactobacillus casei | Idh | CAQ65818.1 | SEQ ID NO: 5 |
| Lactobacillus helveticus | Idh | ABX26516.1 | SEQ ID NO: 6 |
| Lactobacillus delbrueckii subsp. | Idh | KRN37463.1 | SEQ ID NO: 7 |
| bulgaricus | | | |
| Lactobacillus plantarum | Idh | EFK28653.1 | SEQ ID NO: 8 |
| Lactobacillus pentosus | Idh | EIW14906.1 | SEQ ID NO: 9 |
| Lactococcus lactis subsp. lactis | Idh | BAL51029.1 | SEQ ID NO: 10 |

TABLE 2
Examples of O-lactate dehydrogenase (EC 1.1.1.28)

| Microorganism | Gene | GenBank | Protein sequence |
|---|---|---|---|
| Cupriavidus necator H16 | IdhA1 | CAJ92810.1 | SEQ ID NO: 11 |
| Lactobacillus delbrueckii subsp. | IdhA | CAI96942.1 | SEQ ID NO: 12 |
| bulgaricus | | | |
| Escherichia coli str. K-12 substr. | IdhA | 16129341 | SEQ ID NO: 13 |
| MG1655 | | | |
| Bacillus coagulans | IdhA | ADV02473.1 | SEQ ID NO: 14 |

In a particular embodiment, the bacterium is a Cupriavidus necator bacterium, genetically modified at the level of the promoter associated with the gene(s) encoding endogenous L-lactate dehydrogenase and/or endogenous D-lactate dehydrogenase, in order to promote the expression of one and/or the other gene. The inventors have discovered that Cupriavidus necator has genes encoding an endogenous L-lactate dehydrogenase (Idh) and an endogenous D-lactate dehydrogenase (IdhA1), the expression of which is dependent on the culture conditions and is generally nearly zero in nutrient limitation. According to the invention, this bacterium can advantageously be genetically modified at the level of the nucleotide sequence encoding said promoter, in order to overcome this negative regulation and allow the expression of the corresponding genes, even in nutrient limitation. It is in particular possible to modify Cupriavidus necator so as to associate the sequence or sequences encoding endogenous L-lactate dehydrogenase and/or D-lactate dehydrogenase with a constitutive promoter or an inducible promoter.

In an embodiment, the genes encoding endogenous L-lactate dehydrogenase and/or D-lactate dehydrogenase are modified so as to be under the control of a constitutive recombinant promoter (not subject to negative regulation) or a promoter that is inducible in the presence of a particular molecule. By way of example, it is possible to use constitutive promoters such as pLAC, pTAC, pJ5 (Gruber et al., 2014), an inducible promoter such as the pBAD promoter, inducible to arabinose (Grousseau et al., 2014), the pPHAP promoter inducible under phosphate limitation (Barnard et al., 2015), the pCBBL promoter inducible under autotrophic conditions (Lutte et al., 2012) or the pKRrha promoter inducible to rhamnose Sydow et al., 2017).

In a particular embodiment, the bacterium according to the invention is genetically modified to overexpress a gene encoding the expression of a protein having at least 50% homology with SEQ ID NO: 1 (sequence of the L-lactate dehydrogenase of Cupriavidus necator H16), preferentially at least 75%, 80%, 85%, 90%, 95%, 99%. In another embodiment, the bacterium according to the invention is genetically modified to overexpress a gene encoding the expression of a protein having at least 50% homology with SEQ ID NO: 11 (sequence of a D-lactate dehydrogenase of Cupriavidus necator H16), preferentially at least 75%, 80%, 85%, 90%, 95%, 99%.

Alternatively or cumulatively, the bacterium may be genetically modified to overexpress at least one gene encoding an exogenous or heterologous lactate dehydrogenase.

According to the invention, the genome of the bacterium is then modified so as to integrate a nucleic sequence encoding such an exogenous lactate dehydrogenase. Said nucleic sequence may have been introduced into the genome of the bacterium or of one of its ancestors, by means of any suitable molecular cloning method. In the context of the invention, the genome of the bacterium means all the genetic material contained in said bacterium, including extrachromosomal genetic material contained for example in plasmids, episomes, synthetic chromosomes, etc. The introduced nucleic sequence may be a heterologous sequence, i.e. one which does not exist naturally in said bacterium, or a homologous sequence. Advantageously, a transcriptional unit comprising the nucleic sequence of interest is introduced into the genome of the bacterium, placed under the control of one or more promoter(s) and one or more ribosome binding sites. Such a transcriptional unit also comprises, advantageously, the usual sequences such as transcriptional terminators, and, if appropriate, other elements for regulating transcription.

A gene encoding an exogenous lactate dehydrogenase may for example be derived from a bacterium, a fungus, a yeast or a mammal. Preferentially, such a gene is derived from a bacterium and in particular from E. coli, Bacillus coagulans, Pediococcus acidilactici, Streptococcus bovis (Streptococcus equinus), from a lactic acid bacterium, and in particular from Lactobacillus casei, Lactobacillus helveticus, Lactobacillus bulgaricus, Lactobacillus delbrueckii, Lactobacillus plantarum or Lactobacillus pentosus, Lactococcus lactis subsp. lactis. The genes listed in Tables 1 and 2 may also be used to genetically modify a bacterium according to the invention.

In a particular embodiment, the sequence of the heterologous lactate dehydrogenase used to genetically modify the bacterium according to the invention has at least 50% homology with the sequence of the L-lactate dehydrogenase of Pediococcus acidilactici (SEQ ID NO: 2), preferentially at least 60%, 70%, 75%, 80%, 85%, 90%, 95%, 99%, 100%.

In a particular embodiment, the sequence of the heterologous lactate dehydrogenase used to genetically modify the bacterium according to the invention has at least 50% homology with the sequence of the L-lactate dehydrogenase of Streptococcus equinus (Streptococcus bovis) (SEQ ID NO. 3), preferentially at least 60%, 70%, 75%, 80%, 85%, 90%, 95%, 99%, 100%.

In a particular embodiment, the sequence of the heterologous lactate dehydrogenase used to genetically modify the bacterium according to the invention has at least 50% homology with the sequence of the L-lactate dehydrogenase of Bacillus coagulans (SEQ ID NO: 4), preferentially at least 20%, 30%, 40%, 50%, 60%, 70%, 75%, 80%, 85%, 90%, 95%, 99%, 100%.

In a particular embodiment, the sequence of the heterologous lactate dehydrogenase used to genetically modify the bacterium according to the invention has at least 50% homology with the sequence of the L-lactate dehydrogenase of Lactobacillus casei (SEQ ID NO: 5), preferentially at least 20%, 30%, 40%, 50%, 60%, 70%, 75%, 80%, 85%, 90%, 95%, 99%, 100%.

In a particular embodiment, the sequence of the heterologous lactate dehydrogenase used to genetically modify the bacterium according to the invention has at least 50% homology with the sequence of the D-lactate dehydrogenase of Lactobacillus delbrueckii subsp. bulgaricus (SEQ ID NO: 12), preferentially at least 20%, 30%, 40%, 50%, 60%, 70%, 75%, 80%, 85%, 90%, 95%, 99%, 100%.

In a particular embodiment, the sequence of the heterologous lactate dehydrogenase used to genetically modify the bacterium according to the invention has at least 50% homology with the sequence of the D-lactate dehydrogenase of Escherichia coli (SEQ ID NO: 13), preferentially at least 20%, 30%, 40%, 50%, 60%, 70%, 75%, 80%, 85%, 90%, 95%, 99%, 100%.

In a particular embodiment, the sequence of the heterologous lactate dehydrogenase used to genetically modify the bacterium according to the invention has at least 50% homology with the sequence of the D-lactate dehydrogenase of Bacillus coagulans (SEQ ID NO: 14), preferentially at least 20%, 30%, 40%, 50%, 60%, 70%, 75%, 80%, 85%, 90%, 95%, 99%, 100%.

In one embodiment, the bacterium is genetically modified to overexpress at least one gene encoding a L-lactate dehydrogenase (endogenous or heterologous), so as to be able to produce L-lactate. In the case of a bacterium having a gene encoding an endogenous D-lactate dehydrogenase, the expression of said gene is advantageously at least partially inhibited, so as to promote the production of L-lactate only.

Alternatively, the bacterium can be genetically modified to overexpress at least one gene encoding a D-lactate dehydrogenase (endogenous or heterologous) so as to be able to produce D-lactate. In the case of a bacterium having a gene encoding an endogenous L-lactate dehydrogenase, the expression of said gene is advantageously at least partially inhibited, so as to promote the production of D-lactate only.

According to the invention, “inhibition of the expression of a gene” means the fact that said gene is no longer expressed in the bacterium in question or that its expression is reduced, compared with the wild-type bacterium (not genetically modified to inhibit the expression of the gene), leading to the absence of production of the corresponding protein or to a significant reduction in its production, and in particular to a reduction of more than 20%, more preferentially 30%, 40%, 50%, 60%, 70%, 80%, 90%. In one embodiment, the inhibition may be total, i.e. the protein encoded by said gene is no longer produced at all. Inhibition of the expression of a gene can be obtained in particular by deletion, mutation, insertion and/or substitution of one or more nucleotides in the gene in question. Preferentially, the inhibition of the expression of the gene is achieved by total deletion of the nucleotide sequence. According to the invention, any method for inhibiting a gene, known per se to the skilled person and applicable to a bacterium, may be used. For example, inhibition of gene expression can be achieved by homologous recombination (Datsenko et al., 2000; Lodish et al., 2000); random or directed mutagenesis to modify the expression of a gene and/or the activity of the encoded protein (Thomas et al., 2000; Lodish et al., 2000)., 1987); modification of a gene promoter sequence to alter gene expression (Kaufmann et al., 2011); targeting to induce local damage in genomes (TILLING); conjugation, etc. (Kaufmann et al., 2011). Another particular approach is gene inactivation by insertion of a foreign sequence, for example by transposon mutagenesis using mobile genetic elements (transposons), of natural or artificial origin, or for example by insertion of an antibiotic cassette. According to another preferred embodiment, inhibition of gene expression is obtained by knock-out techniques. Inhibition of gene expression can also be achieved by knocking out the gene using interfering RNAs, ribozymes or antisense (Daneholt, 2006. Nobel Prize in Physiology or Medicine). In the context of the present invention, the term “interfering RNA” or “RNAi” refers to any RNAi molecule (for example single-stranded RNA or double-stranded RNA) that can block the expression of a target gene and/or facilitate the degradation of the corresponding mRNA. Gene inhibition can also be achieved by genomic editing methods that allow genetic modifications to be made directly to a given genome via the use of zinc finger nuclease (Kim et al., 1996), of nuclease effectors of the transcription activation type, called “TALEN” (Ousterout et al., 2016), a system combining Cas9 type nucleases with short grouped and regularly spaced palindromic repeats, also called ‘CRISPR’ (Mali et al., 2013), or even meganucleases (Daboussi et al., 2012). Inhibition of gene expression can also be achieved by inactivation of the protein encoded by the said gene. Inhibition of gene expression can also be obtained by deletion, mutation, insertion and/or substitution of one or more nucleotides in the promoter upstream of the gene in question. Inhibition of gene expression can also be obtained by deletion, mutation, insertion and/or substitution of one or more nucleotides in the ribosome binding site upstream of the gene in question.

By working on the genetic modifications to be made to a naturally hydrogen-oxidizing bacterium to enable it to produce lactate from CO₂, the inventors have shown that, in certain bacteria, it is possible to at least partially inhibit a pyruvate degradation pathway that competes with the lactate synthesis pathway, so as to promote the production of lactate from said pyruvate.

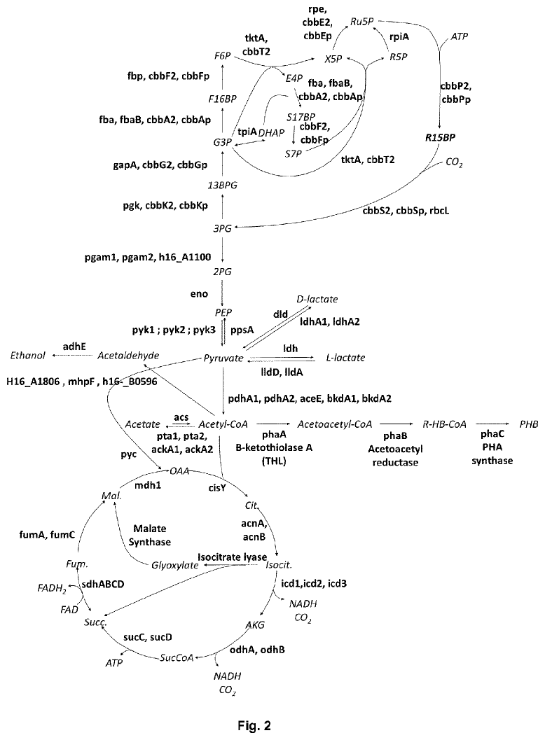

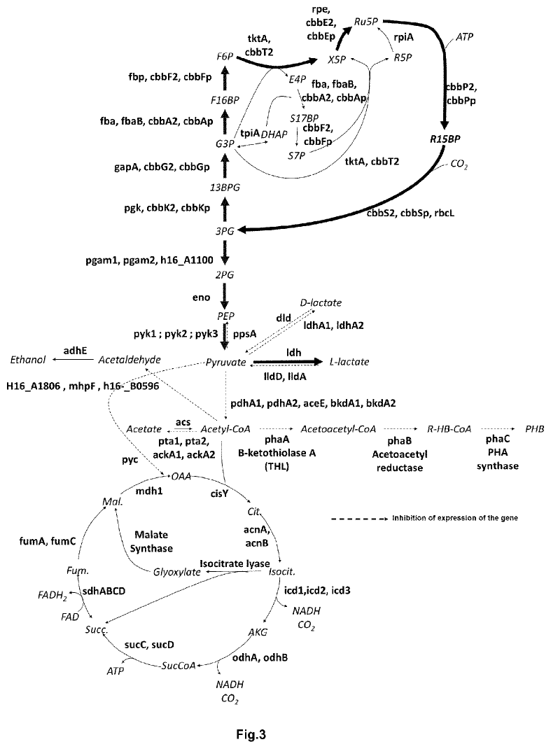

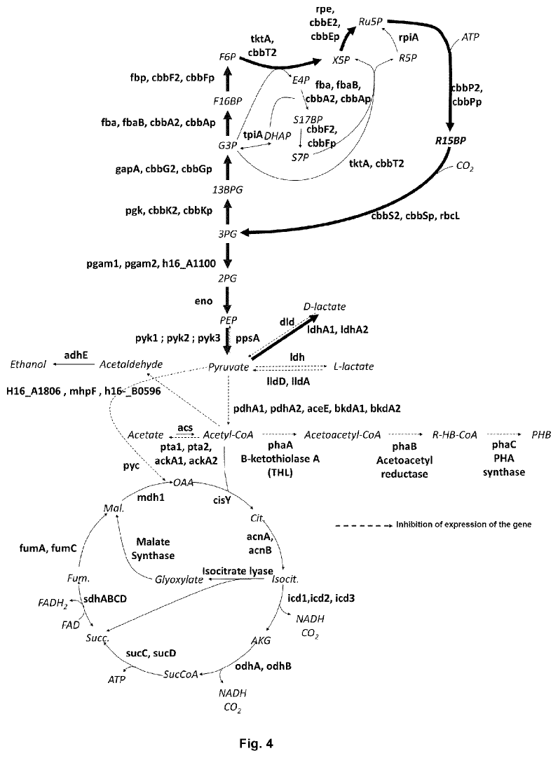

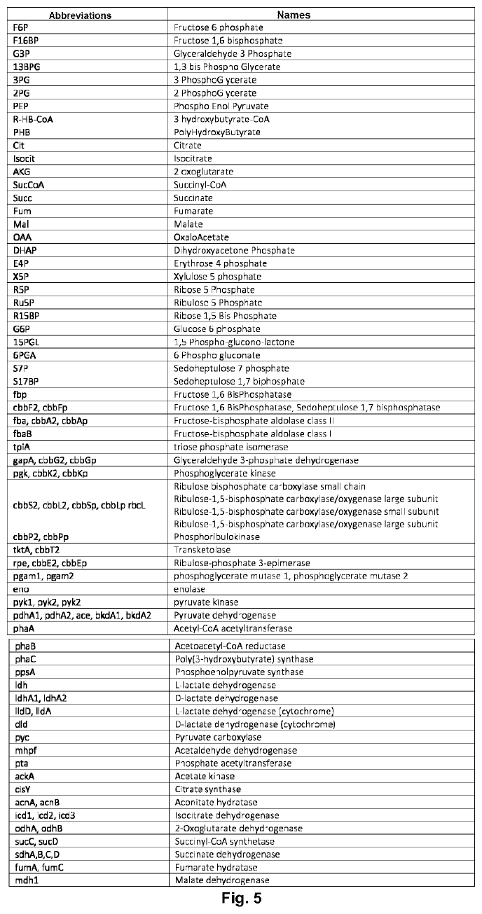

Indeed, certain bacteria which naturally oxidize hydrogen and which can be genetically modified according to the invention, have a route for the synthesis of polyhydroxybutyrate (PHB) from pyruvate. This is notably the case of Cupriavidus necator (FIG. 2). Such a biosynthesis pathway can compete with the lactate synthesis pathway, forcing the consumption of pyruvate in this pathway. Also, in one embodiment, such a bacterium is genetically modified to inhibit at least partially the PHB synthesis pathway (FIG. 3). More particularly, it is possible to at least partially inhibit the expression of at least one gene selected from the genes encoding acetyl-CoA acetyltransferase (EC: 2.3.1.9), acetoacetyl-CoA reductase (EC: 1.1.1.36) and poly(3-hydroxybutyrate) synthase (EC: 2.3.1.-), preferentially the expression of at least two of said genes, more preferentially the expression of said three genes.

In a particular embodiment, the genetically modified bacterium is Cupriavidus necator, in which the expression of at least one and preferentially all three of the genes of the genes encoding a phaA (GenBank: CAJ91322.1), a phaB (GenBank: CAJ92574.1) and a phaC (GenBank: CAJ92572.1) is inhibited (FIG. 2). The inventors have discovered that such a bacterium, also genetically modified to overexpress a lactate dehydrogenase, is capable of producing significant amounts of lactate in the presence of CO₂ as the sole carbon source, the pyruvate produced being not or slightly consumed by the PHB production pathway.

Alternatively or complementarily, it is possible to genetically modify the bacterium so as to at least partially inhibit the expression of a gene encoding a phosphoenolpyruvate synthase (EC: 2.7.9.2), which converts pyruvate to phosphoenol pyruvate (PEP), and/or a pyruvate carboxylase (EC: 6.4.1.1) and/or a pyruvate dehydrogenase complex (EC: 1.2.4.1) and/or a fumarate reductase (EC: 1.3.5.4).

In a particular embodiment, the genetically modified bacterium is Cupriavidus necator, in which the expression of at least one of the genes encoding a ppsa (GenBank: CAJ93138.1), a pyc (GenBank: CAJ92391.1), a pdhA (GenBank: CAJ92510.1) and a sdhABCD (GenBank: CAJ93711.1, CAJ93712.1, CAJ93713.1, CAJ93714.1) is inhibited (FIG. 2). The inventors have discovered that such a bacterium, also genetically modified to overexpress lactate dehydrogenase, is capable of producing significant amounts of lactate in the presence of CO₂ as the sole carbon source, with little or no consumption of the pyruvate produced by these competing pathways.

Alternatively or complementary, the bacterium may be genetically modified so as to at least partially inhibit the conversion pathway of acetyl-CoA to acetate and/or acetaldehyde. To this end, it is possible to at least partially inhibit the expression of at least one gene selected from the genes encoding an acetyl-CoA hydrolase (EC: 3.1.2.1), an acetyl phosphate transferase (EC: 2.3.1.8), an acetate kinase (EC: 2.7.2.1), a propionate CoA-transferase (EC: 2.8.3.1), a succinyl-CoA:acetate CoA-transferase (EC: 2.8.3.18) and an acetaldehyde dehydrogenase (EC: 1.2.1.10).

In a particular embodiment, the genetically modified bacterium is Cupriavidus necator, in which the expression of at least one of the genes encoding an acetyl-CoA hydrolase (GenBank: CAJ96157.1), a phosphate acetyltransferase (pta1, pta2) (GenBank: CAJ96416.1, GenBank: CAJ96653.1), an acetate kinase (ackA, ackA2) (GenBank: CAJ91818.1, GenBank: CAJ96415.1), a propionate CoA-transferase (GenBank: CAJ93797.1), a succinyl-CoA:acetate CoA-transferase (GenBank: CAJ92496.1) and an acetaldehyde dehydrogenase (mhpf) (GenBank: CAJ92911.1) is inhibited (FIG. 2). The inventors have discovered that such a bacterium, also genetically modified to overexpress a lactate dehydrogenase, is capable of producing significant amounts of lactate in the presence of CO₂ as the sole carbon source, with little or no consumption of the pyruvate produced by these competing pathways.

In some cases, naturally hydrogen-oxidizing bacteria have a gene or genes encoding a lactate ferricytochrome C reductase. This is the case in particular of Cupriavidus necator which has three genes IIdD (EC: 1.1.2.3, GenBank: CAJ95257.1), IIdA (EC: 1.1.2.3, GenBank: CAJ96599.1), did (EC: 1.1.2.4, GenBank: CAJ94166.1) encoding such a lactate ferricytochrome C reductase. However, the inventors have discovered that such an enzyme is capable of lowering the level of lactate produced, by converting said lactate into pyruvate. Also, according to the invention, in the case where the bacterium possesses such an endogenous lactate ferricytochrome C reductase, the gene encoding said enzyme is advantageously at least partially inhibited.

The invention proposes advantageously to use a genetically modified bacterium according to the invention to produce lactate from CO₂. Advantageously, a bacterium genetically modified to overexpress an L-lactate dehydrogenase and, if need be, to inhibit a D-lactate dehydrogenase, is used to produce exclusively L-lactate. Similarly, a bacterium genetically modified to overexpress a D-lactate dehydrogenase and, if need be, to inhibit an L-lactate dehydrogenase is used to produce exclusively D-lactate.

The invention more particularly proposes a process for producing lactate from CO₂, according to which a bacterium genetically modified according to the invention is cultured, for example in batch, fed-batch or continuous culture, in the presence of CO₂ and the lactate produced is recovered.

In one embodiment of the process, a base solution is added during fermentation to control the pH. Lactic acid is then present in the fermentation medium in the form of a salt (sodium lactate, potassium lactate, calcium lactate or ammonium lactate, alone or in a mixture, depending on the base chosen to control the pH of the fermentation medium).

The fermentation broth containing the bacteria, the impurities of the fermentation medium (unconsumed proteins and various inorganic salts) and the lactate salt is then treated to separate them. Such a separation step can be carried out using a cell separator such as a centrifuge, a microfiltration or ultrafiltration device. After separation, a concentrated cell biomass and a lactate salt solution are obtained.

It is then possible to proceed to a step of conversion of the lactate salts into lactic acid in free form. This step of recovering the lactic acid from the fermentation medium can be carried out in particular by extracting the lactic acid as such from the fermentation medium, or by acidifying the medium with sulfuric acid.

A step of purification by extraction, esterification, distillation or hydrolysis can then be carried out, in order to obtain lactic acid with a high degree of purity.

According to the invention, the CO₂ source may be pure CO₂ or CO₂ from plants emissions having industrial processes such as petroleum refineries, cement plants, ammonia production, methanol production, etc. CO₂ can also be a fermentation product, a CO₂ enriched gas, an at least partially purified CO₂ gas, a carbonate or bicarbonate solution, and/or formic acid. The CO₂ content of the total gas can be from 10% to 100%. Such CO₂-containing gas can be injected directly or via an intermediate step of CO₂ capture or purification. CO₂ purification can be achieved by chemical treatment, for example in the presence of amines, or by enzymatic treatment using for example a carbonic anhydrase.

According to the invention, the hydrogen source can be a product of steam methane reforming, water electrolysis or be a co-product (“fatal” hydrogen) of industrial processes such as the chlor-alkali process in the preparation of chlorine or waste incineration.

According to the invention, it is possible to eventually predict a growth stage of the bacteria upstream, in particular in the presence of fermentable sugars, such as glucose, fructose or glycerol. It is also possible to grow the bacterium in the presence of CO₂ and another carbon source during the lactate production stage.

### EXAMPLES

### Example 1: Lactate-Producing Strain CN0001

Strain and genetic constructs. For lactate production, a strain Cupriavidus necator H16 PHB-4 (DSM No. 541) is used. This strain does not form poly-β-hydroxy-butyrate (PHB). The construction of the lactate-producing strain CN0001 is carried out according to the following protocol:

A plasmid carrying Cupriavidus necator L-lactate dehydrogenase (Idh, EC: 1.1.1.27) under an arabinose inducible promoter is cloned in one step in vitro using the In-Fusion® assembly protocol (Clontech). Oligonucleotides are synthesized and purified (desalted) by Eurofins Genomics.

The Idh gene is amplified by PCR on the genomic DNA of Cupriavidus necator H16 PHB-4 strain using oligonucleotides 1 (5′ GGATCCAAAC TCGAGTAAGG ATCTCC 3′) and 2 (5′ ATGTATATCT CCTTCTTAAA AGATCTTTTG AATTCC 3′) and the enzyme Phusion High-Fidelity PCRMaster Mix with GC Buffer (New England Biolabs, Evry, France). The PCR product is purified on gel with the Nucleospin gel and PCR clean-up kit (Macherey-Nagel).

The skeletal plasmid pJM3 (Müller et al., 2013) derived from the plasmid pBBR1-MCS2 (Kovach et al., 1995), containing a pBAD promoter from Escherichia coli, is amplified using oligonucleotides 3 (5′ GAAGGAGATA TACATATGAA GATCTCCCTC ACCAGCG 3′) and 4 (5′ CTCGAGTTTG GATCCTCAGG CCGTGGGGAC GGC 3′) and the enzyme Phusion High-Fidelity PCRMaster Mix (New England Biolabs, Evry, France).The PCR product is digested by the Dpnl enzyme (New England Biolabs, Evry, France): 1 μL of Dpnl is added to 50 μL of PCR product, then incubated 15-60 min at 37° C., the enzyme is then inactivated 10 min at 80° C. The PCR product is purified on gel with the Nucleospin gel and PCR clean-up kit (Macherey-Nagel).

An in vitro assembly by In-fusion is performed with the two fragments to obtain the plasmid pJM3-Idh. 5 μL of the assembly product is transformed into chemocompetent Escherichia coli Stellar cells (Clontech). Transformants are selected on a LB/agar medium (Tryptone 10 g/L, yeast extract 5 g/L, NaCl 5 g/L, Sigma-Aldrich, agar 20 g/L) containing 25 μg/mL kanamycin. Selection of positive clones is performed by colony PCR using the DreamTaq Green PCR Master Mix enzyme (Thermo Scientific™). After extraction of the plasmids using the NucleoSpin® Plasmid kit (Macherey-Nagel), the correct insertion of the Idh gene is confirmed by sequencing (Eurofins Genomics) with oligonucleotides 5 (5′ GACGCTTTTT ATCGCAACTC TCTACTG 3′) and 6 (5′ CGAACGCCCT AGGTATAAAC GCAG 3′).

The plasmid pBBAD-Idh is inserted into strain Cupriavidus necator H16 PHB-4 by electroporation (protocol adapted from Taghavi et al., 1994). Cupriavidus necator cells are cultured in TSB medium containing 27.5 g/L tryptic soybean broth (TSB, Becton Dickinson, Sparks, Md., USA, 3 mL in 10 mL tubes) and incubated overnight at 30° C. under vigorous shaking. The next day, 1 mL of the culture is transferred to a 250 mL Erlenmeyer flask containing 25 mL TSB medium and incubated at 30° C., 200 rpm until an OD600nm of 0.5-0.6 is reached. The cells are then harvested and washed twice with 25 mL of cold wash buffer (10% glycerol, 90% water [vol/vol]). The cells rendered by this electro-competent process are then concentrated in the wash solution to obtain an OD600nm of 50 and aliquoted by 150 μL.

An aliquot of competent cells is transformed with 2-5 μL of plasmid DNA (100-150 ng/μL). The DNA/cell mixture contained in a 1.5 mL tube is shaken by gentle tapping (4-5 times). The mixture is placed on ice for 5 min and transferred to a cold electroporation vial (0.2 cm, 10573463, Fisher Scientific, Illkirch). The electroporation is performed with the following settings: voltage 2.5 kV, capacity 25 μF, external resistance 200 Ω. Immediately after electroporation the cells are mixed with 1 mL of SOC medium (tryptone 2%, yeast extract 0.5%, NaCl 10 mM, KCl 2.5 mM, MgCl2 10 mM, MgSO4 10 mM and glucose 20 mM), incubated for 2 h at 30° C. and then plated on Petri dishes maintained at 30° C. containing TSB/Agar medium (20 g/L) with 10 mg/L gentamycin and 200 mg/L kanamycin added).

Strain Cupriavidus necator H16 PHB-4 pJM3-Idh is recovered and named CN0001. Genetic modifications are validated by sequencing.

Media For precultures, the minimum medium A used contains: NaH2PO4, 4.0 g/L; Na2HPO4, 4.6 g/L; K2SO4, 0.45 g/L; MgSO4 0.39 g/L; CaCl2, 0.062 g/L; NH4Cl 0.05% (w/v), trace elements, 1 mL/L (FeSO4.7H2O, 15 g/L; MnSO4.H2O, 2.4 g/L; ZnSO4.7H2O, 2.4 g/L; CuSO4.5H2O, 0.48 g/L in HCM 0.1 M).

For cultures in bioreactors the minimum medium B used contains per liter: MgSO4 7H2O, 0.75 g; phosphate (Na2HPO4.12H2O, 1.5 g; KH2PO4, 0.25 g); nitrilotriacetic acid, 0.285 g; iron(III) ammonium citrate, 0.9 g; CaCl2, 0.015 g; trace elements (H3BO3, 0.45 mg; CoCl2.6H2O, 0.3 mg; ZnSO4.7H2O, 0.15 mg; MnCl2.4H2O, 0.045 mg; Na2MoO4.2H2O, 0.045 mg; NiCl2.6H2O, 0.03 mg; CuSO4, 0.015 mg); kanamycin, 0.1 g.

Precultures An isolated colony of strain CN0001 is used to inoculate the first culture which is grown for 24 h with 10 mL TSB containing 10 mg/L gentamicin and 200 mg/L kanamycin in a 100 mL baffled Erlenmeyer flask. Two further propagation steps are carried out for 12 h each (25 mL and 300 mL minimum medium A in 250 mL and 3 L Erlenmeyer flasks, respectively). Each culture is grown at 30° C. and shaken at 100 rpm in an incubator. This preculture is used to inoculate the culture step in bioreactors.

Culture in bioreactors A fed-batch culture of strain CN0001 is prepared in three phases. The first phase consists of a fructose-controlled growth phase to reach 0.9 g/L biomass at a specific growth rate of 0.16 h⁻¹. After the consumption of fructose, the second phase is performed to allow the adaptation of the cellular metabolism to the gaseous substrates. It is started with a flow of 0.22 L/min of a commercial H2/O2/CO2/N2 mixture (mol %: 60:2:10:28, Air Liquide, Paris, France). The third phase consists of limited nitrogen growth on gaseous substrates coupled with lactate production. This phase is initiated by the addition of 1 g/L L-arabinose to induce the expression of lactate dehydrogenase. Nitrogen is fed from a 56 g/L solution of NH3 to control a residual specific growth rate of 0.02 h⁻¹. This culture is conducted in 1.4 L BDCU B.Braun bioreactors. The temperature is set to 30° C. and pH to 7.0 by adding 2.5 M KOH solution.

Analysis of lactate and metabolites In order to determine the concentrations of organic acids, in particular lactate, the fermentation supernatant is analyzed by HPLC-UV-RI chromatography. The regularly recovered fermentation must (1 mL) is first centrifuged for 10 min at 10 000 g. It is then filtered through 0.45 μm (Minicart RC4, Sartorius). The HPLC system used is a Thermo Scientific UltiMate 3000 HPLC, coupled with a refractometer and UV detector (210 nm). 10 μL of each sample is injected onto an Aminex HPX-87H H+ column, 300 mm×7.8 mm (BioRad). The eluent is an aqueous solution of 4 mM sulfuric acid. The flow rate is fixed at 0.5 mL/min. The oven temperature is 45° C. An isocratic elution is performed. The quantification is carried out using an appropriate standard range. If necessary, the samples are diluted.

Result Strain CN0001 produces 5 to 100 mg/L lactate under these culture conditions.

### Example 2: Construction of a Naturally Hydrogen-Oxidizing Bacterium, Cupriavidus necator, Which is Genetically Modified to Overexpress in Terms of Plasmids an Endogenous Lactate Dehydrogenase and to Produce Lactate From CO₂ (CN0002)

Strain and genetic constructs For lactate production, a strain Cupriavidus necator H16 PHB-4 (DSM No. 541) is used. This strain does not form poly-β-hydroxy-butyrate (PHB).

The construction of the lactate-producing strain CN0002 is carried out according to the following protocol:

A plasmid carrying Cupriavidus necator L-lactate dehydrogenase (Idh, EC: 1.1.1.27) under an arabinose-inducible promoter is cloned in one step in vitro using the In-Fusion® assembly protocol (Clontech). Oligonucleotides are synthesized and purified (desalted) by Eurofins Genomics.

The Idh gene (GenBank: CAJ91814.1) is amplified by PCR on the genomic DNA of Cupriavidus necator H16 strain PHB-4 using oligonucleotides 7 (5′ AAGGAGATAT ACATATGAAG ATCTCCCTCA CCAGCG 3′) and 8 (5′ ACTCGAGTTT GGATCCTCAG GCCGTGGGGA CGGC 3′) and the enzyme Phusion High-Fidelity PCRMaster Mix with GC Buffer (New England Biolabs, Evry, France). The PCR product is purified on gel with the Nucleospin gel and PCR clean-up kit (Macherey-Nagel).

The skeletal plasmid pBADTrfp (Bi et al., 2013) derived from the plasmid pBBR1-MCS (Kovach et al., 1995), containing a pBAD promoter from Escherichia coli, is digested by the restriction enzymes BamHI-HF and Ndel (New England Biolabs, Evry, France) to remove the sequence encoding the RFP protein. The DNA fragment of 5247 base pairs containing the origin of replication pBBR1, the selection gene (kan) and the pBAD promoter is purified on gel with the Nucleospin gel and PCR clean-up kit (Macherey-Nagel).

An in vitro assembly by In-fusion is performed with the two fragments to obtain the plasmid pBAD-Idh(cn). 5 μL of the assembly product are transformed into chemocompetent Escherichia coli Stellar™ cells (Clontech). Transformants are selected on a LB/agar medium (Tryptone 10 g/L, yeast extract 5 g/L, NaCl 5 g/L, Sigma-Aldrich, agar 20 g/L) containing 25 μg/mL kanamycin (Gibco™). Selection of positive clones is performed by colony PCR using the DreamTaq Green PCR Master Mix enzyme (Thermo Scientific™) and oligonucleotides 9 (5′ CGAAGGTGAG CCAGTGTGAC TC 3′) and 10 (5′ CCTGTCGATC CTGCCCAACT AC 3′).

After extraction of the plasmids using the NucleoSpin® Plasmid kit (Macherey-Nagel), the correct insertion of the I.Idh gene is confirmed by sequencing (Eurofins Genomics) with oligonucleotides 9 and 11 (5′ CATTGATTAT TTGCACGGCG TCAC 3′).

The plasmid pBAD-I.Idh(cn) is inserted into an electrocompetent Escherichia coli S17-1 strain by electroporation using a BioRad Gene Pulser electroporator (Datsenko and Wanner, 2000).

Selection of positive clones is performed by colony PCR using the DreamTaq Green PCR Master Mix enzyme (Thermo Scientific™) and oligonucleotides 9 and 10. The clone of interest is isolated on LB/agar medium (Tryptone 10 g/L, yeast extract 5 g/L, NaCl 5 g/L, Sigma-Aldrich, agar 20 g/L) containing 25 μg/mL kanamycin and incubated overnight at 37° C.

Conjugation between Escherichia coli S17-1 cells and those of Cupriavidus necator H16 PHB-4 (DSM No. 541) is performed as follows:

Cupriavidus necator cells are cultured in 3 mL TSB medium containing 27.5 g/L tryptic soybean broth (TSB, Becton Dickinson, Sparks, Md., USA) and incubated 20 h at 30° C. with vigorous shaking. Escherichia coli cells are cultured in 3 mL LB medium (Tryptone 10 g/L, yeast extract 5 g/L, NaCl 5 g/L, Sigma-Aldrich) containing 25 μg/mL kanamycin and incubated overnight at 37° C. with vigorous shaking. The next day, the optical density (OD₆₀₀ₙₘ) is measured for each culture and the equivalent of 1 OD of cells is transferred to a 1.5 mL tube and centrifuged (5 minutes, 3000 rpm). The supernatant is removed and the cells are washed in 1 mL PBS (Sigma-Aldrich). The washing is repeated a second time. 50 μL of Cupriavidus necator cell suspension and 50 μL of Escherichia coli cell suspension are recovered and mixed in a 1.5 mL tube. This mixture is dropped onto a non-selective LB/agar medium containing 2% fructose and incubated for 6 h at 30° C. The cells are then scraped off and resuspended in 1 mL PBS. This suspension is diluted 1:1000 and 100 μL of this dilution is spread on LB/agar selective medium containing 2% fructose, 300 μg/mL kanamycin and 10 μg/mL gentamycin (Sigma-Aldrich). The cells are incubated for 24 h at 30° C. Isolated clones are taken, streaked on the same selective medium and incubated for 24 h at 30° C. Selection of clones containing the plasmid pBAD-Idh(cn) is performed by colony PCR using DreamTaq Green PCR Master Mix (Thermo Scientific™) and oligonucleotides 9 and 10.

Strain Cupriavidus necator H16 PHB-4 pBAD-Idh(cn) is recovered and named CN0002. Genetic modifications are validated by sequencing.

### Example 3: Production of Lactate From Fructose by Culture in Erlenmeyer Flasks of a Genetically Modified, Naturally Hydrogen-Oxidizing Bacterium, Cupriavidus necator (CN0002)

Strain For lactate production, strain CN0002 (Cupriavidus necator H16 PHB-4 pBAD-Idh) is used. In this strain, the C. necator lactate dehydrogenase (LDH) is overexpressed on plasmid via an arabinose-inducible promoter. This strain is naturally resistant to gentamicin and carries plasmid resistance to kanamycin.

Media The rich medium consisted of 27.5% (w/v) soybean trypticase broth (TSB, Becton Dickinson, France). The minimum medium used for Erlenmeyer flask cultures (MMR medium) contained: 4.0 g/L NaH₂PO₄ 2H₂O; 4.6 g/L Na₂HPO₄ 12H₂O; 0.45 g/L K₂SO₄; 0.39 g/L MgSO₄ 7H₂O; 0.062 g/L CaCl₂ 2H₂O and 1 mL/L trace element solution. The trace element solution contained: 15 g/L FeSO₄ 7H₂O; 2.4 g/L MnSO₄ H₂O; 2.4 g/L ZnSO₄ 7H₂O and 0.48 g/L CuSO₄ 5H₂O in 0.1 M HCl. Fructose (20 g/L) was used as the carbon source. NH₄Cl (0.5 g/L) was used as a nitrogen source to achieve a biomass concentration of about 1 g/L. 0.04 g/L NaOH was added.

Inoculation chain A clone of strain CN0002 transplanted from a culture plate was first cultured for 8 h in 5 mL rich medium with gentamicin (10 mg/L) and kanamycin (200 mg/L) (strain-dependent) in culture tubes at 30° C. with shaking (200 rpm). This first preculture was used to inoculate the second preculture at an initial OD₆₀₀ of 0.05 in 25 mL MMR medium in 250 mL baffled Erlenmeyer flasks which were incubated for 20 h at 30° C., 200 rpm. This second preculture was used to inoculate the flask culture into 50 mL MMR medium in the presence of gentamicin (10 mg/L) and kanamycin (200 mg/L) (strain-dependent). The initial target OD₆₀₀ was 0.05.

Flask culture The heterotrophic culture was carried out in a 50 mL volume of MMR in 500 mL baffled Erlenmeyer flasks at a temperature of 30° C. and 200 rpm shaking. A 20 h growth phase was carried out until a biomass of 1 g/L was obtained. After this first growth phase, a modification of the aeration was carried out (2% air, or 0.4% 02), an induction of the LDH promoter was performed by the addition of 1 g/L arabinose (strain-dependent) which led to a lactate production phase lasting 120 h.

Sampling and analysis Protocol 1 mL samples were taken regularly during culture. Growth was monitored by measuring the optical density (OD) at 600 nm; converted to bacterial cell dry weight (gCDW/L) using a calibration curve. Lactate production was analyzed by HPLC as described in Example 1. Samples were centrifuged for 5 min at 13 000 rpm and the supernatants were filtered and analyzed by HPLC. Calibration ranged from 0.1 to 5 g/L in water.

Results The growth of the strain is shown in FIG. 6. During the exponential growth phase, the specific growth rate of CN0002 under heterotrophic conditions reaches μₘₐₓ=0.14h⁻¹. A lactate maximum, i.e. 1.3 g/L, was reached after 139 h of culture as shown in FIG. 6. It should be noted that under the same culture conditions, the Cupriavidus necator H16 PHB-4 strain without overexpression of lactate dehydrogenase does not produce lactate.

### Example 4: Production of Lactate From CO₂ by Fermentation of a Genetically-Modified Naturally Hydrogen-Oxidizing Bacterium, Cupriavidus necator (CN0002)

Strain For lactate production, strain CN0002 (Cupriavidus necator H16 PHB-4 pBAD-Idh) is used. In this strain, the C. necator lactate dehydrogenase (LDH) is overexpressed on plasmid via an arabinose-inducible promoter. This strain is naturally resistant to gentamicin and carries plasmid resistance to kanamycin.

Media The rich medium consisted of 27.5% (w/v) soybean trypticase broth (TSB, Becton Dickinson, France). The minimum medium used for Erlenmeyer flask cultures (MMR medium) contained: 4.0 g/L NaH₂PO₄ 2H₂O; 4.6 g/L Na₂HPO₄ 12H₂O; 0.45 g/L K₂SO₄; 0.39 g/L MgSO₄ 7H₂O; 0.062 g/L CaCl₂ 2H₂O and 1 mL/L trace element solution. The trace element solution contained: 15 g/L FeSO₄ 7H₂O; 2.4 g/L MnSO₄ H₂O; 2.4 g/L ZnSO₄ 7H₂O and 0.48 g/L CuSO₄ 5H₂O in 0.1 M HCl. Fructose (20 g/L) was used as the carbon source. NH₄Cl (0.5 g/L) was used as nitrogen source to achieve a biomass concentration of about 1 g/L. 0.04 g/L NaOH was added.

The minimum medium used in the bioreactor for gas fermentation (FAME medium) consisted of: 0.29 g/L NitriloTriAcetic acid; 0.09 g/L ferric ammonium citrate; 0.75 g/L MgSO₄ 7H₂O; 0.015 g/L CaCl₂ 2H₂O and 1.5 mL/L trace element solution. The composition of the trace element solution was: 0.3 g/L H₃BO₃; 0.2 g/L CoCl₂ 6H₂O; 0.1 g/L ZnSO₄ 7H₂O; 0.03 g/L MnCl₂ 4H₂O; 0.03 g/L Na₂MoO₄ 2H₂O; 0.02 g/L NiCl₂ 6H₂O; 0.01 g/L CuSO₄ 5H₂O. (NH₄)₂SO₄ (1.6 g/L) was used as nitrogen source to achieve a biomass concentration of about 2.5 g/L. The pH was adjusted to 7 with 2.5 M KOH. After autoclaving the medium, a sterile phosphate solution of Na₂HPO_{4 12}H₂O (final concentration 1.6 g/L) and KH₂PO₄ (final concentration 2.9 g/L) was added sterile to the bioreactor (producing about 10 g/L biomass) and a sterile solution of FeSO₄ 7H₂O (10.7 g/L; final concentration 0.032 g/L).

Inoculation chain A clone of strain CN0002 transplanted from a culture plate was first cultured for 24 h in 5 mL TSB medium with gentamicin (10 mg/L) and kanamycin (50 mg/L) (strain-dependent) in 50 mL baffled Erlenmeyer flasks at 30° C. with shaking (110 rpm). The culture medium was then centrifuged for 10 min at 1900 g. The cells were resuspended in 5 mL MMR medium, and were used to inoculate the 45 mL MMR medium with 5.5 mg/L gentamicin and 100 mg/L kanamycin (strain-dependent) in 500 mL baffled Erlenmeyer flasks which were incubated for 20-24 hat 30° C., 110 rpm. The culture medium was centrifuged for 5 min at 4000 g and the cells were resuspended in 30 mL FAME medium and used to inoculate the 300 mL FAME medium (with 100 mg/L kanamycin if necessary) into the gas bioreactor. The initial target OD₆₀₀ was 0.2.

Gas bioreactor The autotrophic culture was carried out in a gas bioreactor with a working volume of 330 mL. The temperature was set at 30° C., pH 7. Pressure and shaking speed were set according to the requirements of the culture. The gas flows were individually controlled (CO₂, H₂, air). A gas analyzer was used to analyze the output gases (% O₂ and CO₂) and a volumeter to measure the total output gas flows. After 53 hours of growth, approximately 3 g/L biomass was reached, the dissolved oxygen concentration dropped to 0 and 160 mg/L lactate was produced.

Sampling and analysis protocol Samples (approximately 1 mL) were taken regularly (every 2-3 hours) by taking a sample through a septum with a syringe and needle. Growth was followed by measurement of optical density (OD) at 600 nm; converted to bacterial cell dry weight (gCDW/L) according to a calibration curve. Lactate production was analyzed by HPLC/HPAIC as described in Example 1. The samples were centrifuged for 3 min at 13 000 rpm and the supernatants were analyzed. For HPLC analysis, the supernatants of the samples were filtered out before analysis. Calibration ranged from 0.1 to 5 g/L in water.

Fermentation Lactate production by strain CN0002 was characterized in bioreactor under autotrophic conditions. The growth of the strain is shown in FIG. 7. During the exponential growth phase, the specific growth rate of CN0002 under autotrophic conditions reaches μₘₐₓ=0.14 h⁻¹. A lactate maximum, i.e. 160 mg/L, was reached after 53 h of culture as shown in FIG. 7.

### Example 5: Construction and Evaluation of a Genetically Modified Strain of Cupriavidus necator in Which a Heterologous Streptococcus bovis Lactate Dehydrogenase is Overexpressed (CN0003)

Strain and genetic constructs. For lactate production, a strain Cupriavidus necator H16 PHB-4 (DSM No. 541) is used. This strain does not form poly-β-hydroxy-butyrate (PHB). The construction of the lactate-producing strain CN0003 is carried out according to the following protocol:

A plasmid carrying the L-lactate dehydrogenase (Idh, EC: 1.1.1.27) of Streptococcus bovis (ATCC 33317) under an arabinose inducible promoter is cloned in one step in vitro via the In-Fusion® assembly protocol (Clontech). The oligonucleotides are synthesized and purified (desalted) by Eurofins Genomics.

The Idh gene (GenBank: KFN85486.1) is recoded according to the codon usage bias described for Cupriavidus necator H16 and synthetized in vitro (GenScript®). It is amplified by PCR using oligonucleotides 12 (5′ AAGGAGATAT ACATATGACC GCGACCAAGC AGCAC 3′) and 13 (5′ ACTCGAGTTT GGATCCTCAG TTCTTGCAGG CCGACGCGA 3′) and the enzyme Phusion High-Fidelity PCRMaster Mix with GC Buffer (New England Biolabs, Evry, France). The PCR product is purified on gel with the Nucleospin gel and PCR clean-up kit (Macherey-Nagel).

The skeletal plasmid pBADTrfp (Bi et al., 2013) derived from the plasmid pBBR1-MCS (Kovach et al., 1995), containing a pBAD promoter from Escherichia coli, is digested by the restriction enzymes BamHl-HF and Ndel (New England Biolabs, Evry, France) to remove the sequence encoding the RFP protein. The DNA fragment of 5247 base pairs containing the origin of replication pBBR1, the selection gene (kan) and the pBAD promoter is purified on gel with the Nucleospin gel and PCR clean-up kit (Macherey-Nagel).

An in vitro assembly by In-fusion is performed with the two fragments to obtain the plasmid pBAD-Idh(sb). 5 μL of the assembly product is transformed into chemocompetent Escherichia coli Stellar™ cells (Clontech). Transformants are selected on a LB/agar medium (Tryptone 10 g/L, yeast extract 5 g/L, NaCl 5 g/L, Sigma-Aldrich, agar 20 g/L) containing 25 μg/mL kanamycin (Gibco™). Selection of positive clones is performed by colony PCR using the DreamTaq Green PCR Master Mix enzyme (Thermo Scientific™) and oligonucleotides 9 and 14 (5′ GGAGCTGGGC ATCATCGAGA TC 3′). After extraction of the plasmids using the NucleoSpin® Plasmid kit (Macherey-Nagel), the correct insertion of the Idh gene is confirmed by sequencing (Eurofins Genomics) with oligonucleotides 9 and 11.

The plasmid pBAD-Idh(sb) is inserted into an electrocompetent Escherichia coli S17-1 strain by electroporation using a BioRad Gene Pulser electroporator (Datsenko and Wanner, 2000).

Selection of positive clones is performed by colony PCR using the DreamTaq Green PCR Master Mix enzyme (Thermo Scientific™) and oligonucleotides 9 and 14. The clone of interest is isolated on LB/agar medium (Tryptone 10 g/L, yeast extract 5 g/L, NaCl 5 g/L, Sigma-Aldrich, agar 20 g/L) containing 25 μg/mL kanamycin and incubated overnight at 37° C.

Conjugation between Escherichia coli S17-1 and Cupriavidus necator H16 PHB-4 (DSM No. 541) cells is performed as follows:

Cupriavidus necator cells are cultured in 3 mL TSB medium containing 27.5 g/L tryptic soybean broth (TSB, Becton Dickinson, Sparks, Md., USA) and incubated 20 h at 30° C. with vigorous shaking. Escherichia coli cells are cultured in 3 mL LB medium (Tryptone 10 g/L, yeast extract 5 g/L, NaCl 5 g/L, Sigma-Aldrich) containing 25 μg/mL kanamycin and incubated overnight at 37° C. with vigorous shaking. The next day, the optical density (O₆₀₀ₙₘ) is measured for each culture and the equivalent of 1 OD of cells is transferred to a 1.5 mL tube and centrifuged (5 minutes, 3000 rpm). The supernatant is removed and the cells are washed in 1 mL PBS (Sigma-Aldrich). The washing is repeated a second time. 50 μL of Cupriavidus necator cell suspension and 50 μL of Escherichia coli cell suspension are recovered and mixed in a 1.5 mL tube. This mixture is deposited as a drop on a non-selective LB/agar medium containing 2% fructose and incubated for 6 h at 30° C. The cells are then scraped off and resuspended in 1 mL PBS. This suspension is diluted 1:1000 and 100 μL of this dilution is spread on LB/agar selective medium containing 2% fructose, 300 μg/mL kanamycin and 10 μg/mL gentamycin (Sigma-Aldrich). The cells are incubated for 24 h at 30° C. Isolated clones are taken, streaked on the same selective medium and incubated for 24 h at 30° C. Selection of clones containing the plasmid pBAD-Idh(sb) is performed by colony PCR using DreamTaq Green PCR Master Mix (Thermo Scientific™) and oligonucleotides 9 and 14.

Cupriavidus necator H16 PHB-4 pBAD-Idh(sb) strain Cupriavidus necator H16 PHB-4 pBAD-Idh(sb) is recovered and named CN0003.

Evaluation of strain CN0003 in fructose The lactate-on-fructose production of Cupriavidus necator H16 PHB-4 pBAD-Idh(sb) strain (CN0003) was evaluated using the method described in Example 3. Under these culture conditions, strain CN0003 produces 1 g/L lactate after 140 h of culture.

Extrapolation of CN0003 lactate production from CO₂. The lactate production of strain CN0003 was not derived from CO₂. However, the fructose evaluation showed that strain CN0003 produced 23% less lactate than strain CN0002 under the same culture conditions. We can extrapolate that from CO₂ as described in Example 4, strain CN0003 would produce 23% less lactate from CO₂ than strain CN0002.

### Example 6: Construction of a Genetically Modified Cupriavidus necator Strain in Which a Polyhydroxybutyrate (PHB) Biosynthetic Pathway is Deleted and in Which an Endogenous Lactate Dehydrogenase is Overexpressed (CN0004)

Strain For lactate production, the strain Cupriavidus necator H16 (ATCC 17699) is used. The deletion of the polyhydroxybutyrate (PHB) biosynthesis pathway operon is inhibited by insertion of an endogenous lactate dehydrogenase.

Genetic constructs The construction of the lactate-producing strain CN0004 is carried out according to the following protocol:

A plasmid carrying Cupriavidus necator L-lactate dehydrogenase (Idh, EC: 1.1.1.27) under an arabinose-inducible promoter as well as the sequences of the upstream and downstream homology zones of the phaCAB operon is cloned in one step in vitro via the NEBuilder® HiFi DNA Assembly Cloning protocol (New England Biolabs, Evry, France). Oligonucleotides are synthesized and purified (desalted) by Eurofins Genomics.

The Idh gene is amplified by PCR on the genomic DNA of the Cupriavidus necator H16 strain using oligonucleotides 15 (5′ AGACAATCAA ATCTTTACAC TTTATGCTTC CGGCTCGTAT GTTGTGTGGA ATTGTGAGCG GATAACAATT TCACACAGGA AACAGCTATG AAGATCTCCC TCACCAGCGC CC 3′) having a 5′ overlap of 13 base pairs with the 3′ end of the upstream homology zone of the phaCAB operon and the pLac promoter sequence (in italics) from pJQ200mp18 (Quandt and Hynes, 1993) and 16 (5′ CCAGGCCGGC AGGTCAGGCC GTGGGGACGG CCA 3′) having an overlap area of 13 base pairs with the 5′ end of the downstream homology area of the phaCAB operon and the enzyme KOD Hot Start DNA Polymerase (Novagen) with 5% DMSO. The PCR product is purified on gel with the Nucleospin gel and PCR clean-up kit (Macherey-Nagel).

The sequence of the zone of upstream homology of the phaCAB operon is amplified by PCR on the genomic DNA of the Cupriavidus necator H16 strain using oligonucleotides 17 (5′ GCATGCCTGC AGGTCGACTC TAGAGGGTCG CTTCTACTCC TATCG 3′) having at the 5′ end an overlapping zone of 25 pairs of bases with the 3′ end of the plasmid pJQ200mpTet and 18 (5′ CATAAAGTGT AAAGATTTGA TTGTCTCTCT GCC 3′) having an overlapping region of 13 base pairs with the 5′ end of the sequence of the pLac promoter and the enzyme KOD Hot Start DNA Polymerase (Novagen) with 5% DMSO. The PCR product is purified on gel with the Nucleospin gel and PCR clean-up kit (Macherey-Nagel).

The sequence of the downstream homology region of the phaCAB operon is amplified by PCR on the genomic DNA of the Cupriavidus necator H16 strain using oligonucleotides 19 (5′ CCCCACGGCC TGACCTGCCG GCCTGGTTCA ACC 3′) having at the 5′ end an overlapping region of 13 base pairs with the 3′ end of the sequence of the Idh gene of strain Cupriavidus necator H16 and 20 (5′ TACGAATTCG AGCTCGGTAC CCGGGTTCTG GATGTCGATG AAGGCCTG 3′) having at the 5′ end an overlapping region of 25 base pairs with the 5′ end of the plasmid pJQ200mpTet and the enzyme KOD Hot Start DNA Polymerase (Novagen) with 5% DMSO. The PCR product is purified on gel with the Nucleospin gel and PCR clean-up kit (Macherey-Nagel).

The skeletal plasmid pJQ200mpTet is a derivative of the plasmid pJQ200mp18 (Quandt and Hynes, 1993) where the gentamicin resistance gene has been replaced by the tetracycline resistance gene. The skeletal plasmid pJQ200mpTet is cleaved by the restriction enzyme BamHI-HF (New England Biolabs, Evry, France). The digestion product is purified on gel with the Nucleospin gel and PCR clean-up kit (Macherey-Nagel).

An in vitro assembly by NEBuilder® HiFi DNA Assembly Cloning (New England Biolabs, Evry, France) is performed with the four fragments to obtain the plasmid pJQ200mp-ΔphaCABΩpLac L-Idh. 2 μof the assembly product is transformed into chemocompetent NEB 5-alpha competent Escherichia coli cells (New England Biolabs, Evry, France). Transformants are selected on a LB/agar medium (Tryptone 10 g/L, yeast extract 5 g/L, NaCl 5 g/L, Sigma-Aldrich, agar 20 g/L) containing 15 μg/mL tetracycline. Selection of positive clones is performed by colony PCR using the DreamTaq Green PCR Master Mix enzyme (Thermo Scientific™). After extraction of the plasmids using the NucleoSpin® Plasmid kit (Macherey-Nagel), the correct insertion of the Idh gene is performed, of the sequence of the upstream homology zone and the sequence of the downstream homology zone is confirmed by sequencing (Eurofins Genomics) with oligonucleotides 21 (5′ TGCAAGGCGA TTAAGTTG 3′). 22 (5′ CATGCAAAGT GCCGGCCAGG 3′), 23 (5′ CTGCACGAAC ATGGTGCTGG CT 3′) and 24 (5′ CTGGCACGAC AGGTTTCCCG A 3′).

The plasmid pJQ200mp-ΔphaCABΩpLac L-Idh is inserted into an electro-competent Escherichia coli S17-1 strain by electroporation using a BioRad Gene Pulser electroporator (Datsenko and Wanner, 2000).

Selection of positive clones is performed by colony PCR using the DreamTaq Green PCR Master Mix enzyme (Thermo Scientific™). The clone of interest is isolated on LB/agar medium (Tryptone 10 g/L, yeast extract 5 g/L, NaCl 5 g/L, Sigma-Aldrich, agar 20 g/L) containing 15 μg/mL tetracycline and incubated overnight at 37° C.

The conjugation between Escherichia coli S17-1 and Cupriavidus necator H16 (ATCC 17699) cells was adapted from the Lenz et al., 1994 protocol. The sucrose selection method (15% sucrose used) with SacB allowed precise deletion of the phaCAB operon and insertion of pLac L-Idh. After the first homologous recombination, the clones were validated by colony PCR using the DreamTaq Green PCR Master Mix enzyme (Thermo Scientific™). After the second homologous recombination, the selection of positive clones from tetracycline-sensitive clones is performed by colony PCR using the enzyme KOD Xtreme™ Hot Start DNA Polymerase (Novagen) (Krauβe et al., 2009).

The strain Cupriavidus necator H16 ΔphaCABΩpLAC_L-Idh C. necator is recovered and named CN0004. Genetic modifications are validated by sequencing.

Evaluation of strain CN0004 in fructose The lactate-on-fructose production of Cupriavidus necator H16 strain ΔphaCABΩpLAC_L-Idh C. necator (CN0004) was evaluated using the method described in Example 3. Under these culture conditions, strain CN0004 produces 1.5 g/L lactate after 140 h of culture.

Extrapolation of CN0004 lactate production from CO₂ The lactate production of strain CN0004 was not derived from CO₂. However, the fructose evaluation showed that CN0004 produced 25% more lactate than strain CN0002 under the same culture conditions. We can extrapolate that from CO₂ as described in Example 4, strain CN0004 would produce 25% more lactate from CO₂ than strain CN0002.

### Example 7: Construction of a Genetically Modified Cupriavidus necator Strain in Which a Polyhydroxybutyrate (PHB) Biosynthetic Pathway is Deleted, an Endogenous Lactate Dehydrogenase is Overexpressed and a Pyruvate Carboxylase is Deleted (CN0005)

Strain and genetic constructs The construction of the lactate-producing strain CN0005 is carried out according to the following protocol:

A plasmid carrying the sequences of the upstream and downstream homology zones of the gene encoding pyruvate carboxylase (pyc) from Cupriavidus necator is cloned in one step in vitro using the NEBuilder® HiFi DNA Assembly Cloning protocol (New England Biolabs, Evry, France). Oligonucleotides are synthesized and purified (desalted) by Eurofins Genomics.

The sequence of the zone of upstream homology of the gene encoding pyc is amplified by PCR on the genomic DNA of the Cupriavidus necator H16 strain using oligonucleotides 25 (5′ AGATCCTTTA ATTCGAGCTC GGTACCGCAT GGCCAAGGTG GAAGAG 3′) having at the 5′ end an overlapping zone of 25 base pairs with the 3′ end of the plasmid pLO3 and 26 (5′ TGCCGGCCAA CGTCACATGG GATGCAGGGA AGCGAAC 3′) having at the 5′ end an overlapping region of 15 base pairs with the 5′ end of the downstream homology region of the gene encoding pyc and the enzyme KOD Hot Start DNA Polymerase (Novagen) with 5% DMSO. The PCR product is purified on gel with the Nucleospin gel and PCR clean-up kit (Macherey-Nagel).

The sequence of the downstream homology region of the gene encoding pyc is amplified by PCR on the genomic DNA of the Cupriavidus necator H16 strain using oligonucleotides 27 (5′ TCCCTGCATC CCATGTGACG TTGGCCGGCA GGG 3′) having at the 5′ end an overlapping region of 15 base pair with the 3′ end of the upstream homology region of the gene encoding pyc and 28 (5′ ACTTAATTAA GGATCCGGCG CGCCCCCCGG GCTGATAGTT CTTCAACACC AGCAGTC 3′) having at the 5′ end an overlapping region of 31 base pairs with the 5′ end of plasmid pLO3 and the enzyme KOD Hot Start DNA Polymerase (Novagen) with 5% DMSO. The PCR product is purified on gel with the Nucleospin gel and PCR clean-up kit (Macherey-Nagel).

The skeletal plasmid pLO3 (Lenz and Friedrich, 1998) is cleaved by the restriction enzyme Xmal-HF (New England Biolabs, Evry, France). The digestion product is purified on gel with the Nucleospin gel and PCR clean-up kit (Macherey-Nagel).

An in vitro assembly by NEBuilder® HiFi DNA Assembly Cloning (New England Biolabs, Evry, France) is performed with the three fragments to obtain the plasmid pLO3-Δpyc. 2 μL of the assembly product is transformed into chemocompetent, NEB 5-alpha competent Escherichia coli cells (New England Biolabs, Evry, France). Transformants are selected on a LB/agar medium (Tryptone 10 g/L, yeast extract 5 g/L, NaCl 5 g/L, Sigma-Aldrich, agar 20 g/L) containing 15 μg/mL tetracycline. Selection of positive clones is performed by colony PCR using the DreamTaq Green PCR Master Mix enzyme (Thermo Scientific™). After extraction of the plasmids using the NucleoSpin® Plasmid kit (Macherey-Nagel), deletion of the pyc gene is confirmed by sequencing (Eurofins Genomics) with oligonucleotides 29 (5′ GCAAACAAAC CACCGCTGGT 3′), 30 (5′ CGCCATATCG GATGCCGTTC 3′) and 31 (5′ TAGCAGCACG CCATAGTGAC 3′).

The plasmid pLO3-Δpyc is inserted into an electrocompetent Escherichia coli S17-1 strain by electroporation using a BioRad Gene Pulser electroporator (Datsenko and Wanner, 2000).

Selection of positive clones is performed by colony PCR using the DreamTaq Green PCR Master Mix enzyme (Thermo Scientific™). The clone of interest is isolated on LB/agar medium (Tryptone 10 g/L, yeast extract 5 g/L, NaCl 5 g/L, Sigma-Aldrich, agar 20 g/L) containing 15 μg/mL tetracycline and incubated overnight at 37° C.

The conjugation between Escherichia coli S17-1 cells and those of strain CN0004 was adapted from the Lenz et al., 1994 protocol. The sucrose selection method (15% sucrose used) with SacB allowed to precisely delete the gene encoding pyc. After the first homologous recombination, the clones were validated by colony PCR using the DreamTaq Green PCR Master Mix enzyme (Thermo Scientific™). After the second homologous recombination, the selection of positive clones from tetracycline-sensitive clones is performed by colony PCR using the KOD Xtreme™ Hot Start DNA Polymerase (Novagen) enzyme.

The strain Cupriavidus necator H16 Δpyc ΔphaCABΩpLAC_L-Idh C. necator is recovered and named CN0005. Genetic modifications are validated by sequencing.

Evaluation of strain CN0005 in fructose Lactate production on fructose of strain Cupriavidus necator H16 Δpyc ΔphaCABΩpLAC_L-Idh C. necator (CN0005) was evaluated using the method described in Example 3. Under these culture conditions, strain CN0005 produces 21% more lactate than strain CN0004 after 51 h of culture.

Extrapolation of CN0005 lactate production from CO₂ The lactate production of strain CN0005 was not derived from CO₂. However, the fructose evaluation showed that CN0005 produced 21% more lactate than strain CN0004 under the same culture conditions. We can extrapolate that from CO₂ as described in Example 4, strain CN0005 would produce 25% more lactate from CO₂ than strain CN0004.

### Example 8: Construction of a Genetically Modified Cupriavidus necator Strain in Which a Polyhydroxybutyrate (PHB) Biosynthetic Pathway is Deleted, an Endogenous Lactate Dehydrogenase is Overexpressed and a Pyruvate Dehydrogenase is Deleted (CN0006)

Strain and genetic constructs The construction of the lactate-producing strain CN0006 is carried out according to the following protocol:

A plasmid carrying the sequences of the upstream and downstream homology zones of the gene encoding the E1 component of the pyruvate dehydrogenase (pdhA2) of Cupriavidus necator is cloned in one step in vitro using the NEBuilder® HiFi DNA Assembly Cloning protocol (New England Biolabs, Evry, France). Oligonucleotides are synthesized and purified (desalted) by Eurofins Genomics.

The sequence of the upstream homology zone of the gene encoding pdhA2 is amplified by PCR on the genomic DNA of the Cupriavidus necator H16 strain using oligonucleotides 32 (5′ TCCTTTAATT CGAGCTCGGT ACCCGGGTGC GTAATCCACT TCCAG 3′) having at the 5′ end an overlapping zone of 22 base pairs with the 3′ end of plasmid pLO3 and 33 (5′ CCCATCGTTC ACACGGCAAG TCTCCGTTAA GGAATTC 3′) having at the 5′ end an overlapping region of 11 base pairs with the 5′ end of the downstream homology region of the gene encoding pdhA2 and the enzyme KOD Hot Start DNA Polymerase (Novagen) with 5% DMSO. The PCR product is purified on gel with the Nucleospin gel and PCR clean-up kit (Macherey-Nagel).

The sequence of the downstream homology region of the gene encoding pdhA2 is amplified by PCR on the genomic DNA of the Cupriavidus necator H16 strain using oligonucleotides 34 (5′ GACTTGCCGT GTGAACGATG GGCCATCGGG CA 3′) having at the 5′ end an overlapping region of 11 base pairs with the end 3′ of the upstream homology region of the gene encoding pdhA2 and 35 (5′ TAAGGATCCG GCGCGCCCCC GGGTTGAGCA GGATCACGTC GATCC 3′) having at the 5′ end an overlapping region of 22 base pairs with the 5′ end of plasmid pLO3 and the enzyme KOD Hot Start DNA Polymerase (Novagen) with 5% DMSO. The PCR product is purified on gel with the Nucleospin gel and PCR clean-up kit (Macherey-Nagel).

The skeletal plasmid pLO3 (Lenz and Friedrich, 1998) is cleaved by the restriction enzyme Xmal-HF (New England Biolabs, Evry, France). The digestion product is purified on gel with the Nucleospin gel and PCR clean-up kit (Macherey-Nagel).

An in vitro assembly by NEBuilder® HiFi DNA Assembly Cloning (New England

Biolabs, Evry, France) is performed with the three fragments to obtain the plasmid pLO3-ΔpdhA2. 2 μL of the assembly product is transformed into chemocompetent, NEB 5-alpha competent Escherichia coli cells (New England Biolabs, Evry, France). Transformants are selected on a LB/agar medium (Tryptone 10 g/L, yeast extract 5 g/L, NaCl 5 g/L, Sigma-Aldrich, agar 20 g/L) containing 15 μg/mL tetracycline. Selection of positive clones is performed by colony PCR using the DreamTaq Green PCR Master Mix enzyme (Thermo Scientific™). After extraction of the plasmids using the NucleoSpin® Plasmid kit (Macherey-Nagel), the deletion of the pdhA2 gene is confirmed by sequencing (Eurofins Genomics) with oligonucleotides 36 (5′ TAATCCACTT CCAGCGCGAT AAG 3′), 37 (5′ CCTGAAGTCT CCGCGATAAC 3′), 38 (5′ GTTCGAAGCC ACCGAGTATG AC 3′) and 29 (5′ GCAAACAAAC CACCGCTGGT 3′).

The plasmid pLO3-ΔpdhA2 is inserted into an electrocompetent Escherichia coli S17-1 strain by electroporation using a BioRad Gene Pulser electroporator (Datsenko and Wanner, 2000).

Selection of positive clones is performed by colony PCR using the DreamTaq Green PCR Master Mix enzyme (Thermo Scientific™). The clone of interest is isolated on LB/agar medium (Tryptone 10 g/L, yeast extract 5 g/L, NaCl 5 g/L, Sigma-Aldrich, agar 20 g/L) containing 15 μg/mL tetracycline and incubated overnight at 37° C.

The conjugation between Escherichia coli S17-1 cells and those of strain CN0004 was adapted from the Lenz et al., 1994 protocol. The sucrose selection method (15% sucrose used) with SacB allowed to precisely delete the gene encoding pdhA2. After the first homologous recombination, the clones were validated by colony PCR using the DreamTaq Green PCR Master Mix enzyme (Thermo Scientific™). After the second homologous recombination, the selection of positive clones among the tetracycline-sensitive clones is performed by colony PCR using the KOD Xtreme™ Hot Start DNA Polymerase (Novagen) enzyme.

The Cupriavidus necator H16 strain ΔpdhA2 ΔphaCABΩpLAC_L-Idh C. necator is recovered and named CN0006. Genetic modifications are validated by sequencing.

Evaluation of strain CN0006 in fructose Lactate production on fructose of Cupriavidus necator strain H16 ΔpdhA2 ΔphaCABΩpLAC_L-Idh C. necator (CN0006) was evaluated using the method described in Example 3. Under these culture conditions, strain CN0006 produces 13% more lactate than strain CN0004 after 51 hours of culture.

Extrapolation of CN0006 lactate production from CO₂ The lactate production of strain CN0006 was not derived from CO₂. However, the fructose evaluation showed that strain CN0006 produced 13% more lactate than strain CN0004 under the same culture conditions. We can extrapolate that from CO₂ as described in Example 4, strain CN0006 would produce 13% more lactate from CO₂ than strain CN0004.

### Example 9: Construction of a Genetically Modified Cupriavidus necator Strain in Which a Polyhydroxybutyrate (PHB) Biosynthetic Pathway is Deleted, an Endogenous Lactate Dehydrogenase is Overexpressed and in Which an Acetyltransferase Phosphate and an Acetate Kinase Phosphoenolpyruvate Synthase are Deleted (CN0007)

### Strain and Genetic Constructs

The construction of the lactate-producing strain CN0007 is carried out according to the following protocol:

A plasmid carrying the sequences of the upstream and downstream homology zones of the operon encoding the acetate kinase (ackA) and phosphotransacetylase (pta1) genes of Cupriavidus necator is cloned in one step in vitro using the NEBuilder® HiFi DNA Assembly Cloning protocol (New England Biolabs, Evry, France). Oligonucleotides are synthesized and purified (desalted) by Eurofins Genomics.

The sequence of the upstream homology zone of the gene encoding pta1 is amplified by PCR on the genomic DNA of the Cupriavidus necator H16 strain using oligonucleotides 39 (5′ TCCTTTAATT CGAGCTCGGT ACGTGTCCAA TGAGATGACA GCACG 3′) having at the 5′ end an overlapping zone of 22 base pairs with the 3′ end of the plasmid pLO3 and 40 (5′ TGTAGCGGTG GTGCGTCAGG GTCGTCGGTG 3′) having at the 5′ end an overlapping region of 11 base pairs with the 5′ end of the downstream homology region of the gene encoding ackA and the enzyme KOD Hot Start DNA Polymerase (Novagen) with 5% DMSO. The PCR product is purified on gel with the Nucleospin gel and PCR clean-up kit (Macherey-Nagel).

The sequence of the downstream homology region of the gene encoding ackA is amplified by PCR on the genomic DNA of the Cupriavidus necator H16 strain using oligonucleotides 41 (5′ ACCCTGACGC ACCACCGCTA CAGCCGACCA AG 3′) having at the 5′ end an overlapping region of 11 base pairs with the end 3′ of the upstream homology region of the gene encoding pta1 and 42 (5′ TAAGGATCCG GCGCGCCCCC GGGCTGATAC

GTTCACGCAT AGTGGTC 3′) having at the 5′ end an overlapping region of 22 base pairs with the 5′ end of the plasmid pLO3 and the enzyme KOD Hot Start DNA Polymerase (Novagen) with 5% DMSO. The PCR product is purified on gel with the Nucleospin gel and PCR clean-up kit (Macherey-Nagel).

The skeletal plasmid pLO3 (Lenz and Friedrich, 1998) is cleaved by the restriction enzyme Xmal-HF (New England Biolabs, Evry, France). The digestion product is purified on gel with the Nucleospin gel and PCR clean-up kit (Macherey-Nagel).

An in vitro assembly by NEBuilder® HiFi DNA Assembly Cloning (New England Biolabs, Evry, France) is performed with the three fragments to obtain the plasmid pLO3-Δpta1-ackA. 2 μL of the assembly product is transformed into chemocompetent, NEB 5-alpha competent Escherichia coli cells (New England Biolabs, Evry, France). Transformants are selected on a LB/agar medium (Tryptone 10 g/L, yeast extract 5 g/L, NaCl 5 g/L, Sigma-Aldrich, agar 20 g/L) containing 15 μg/mL tetracycline. Selection of positive clones is performed by colony PCR using the DreamTaq Green PCR Master Mix enzyme (Thermo Scientific™). After extraction of the plasmids using the NucleoSpin® Plasmid kit (Macherey-Nagel), the deletion of the ackA-pta1 operon is confirmed by sequencing (Eurofins Genomics) with oligonucleotides 43 (5′ GACTTCCGGC AGGTCATGC 3′), 44 (5′ CAGTTGTTGC GCTGCAGTCA T 3′), 45 (5′ GCCAAGCCGG AACGCGTC 3′) and 46 (5′ GATGGTGGCA CGATGTTCAC 3′).

The plasmid pLO3-Δpta1-ackA is inserted into an electrocompetent Escherichia coli S17-1 strain by electroporation using a BioRad Gene Pulser electroporator (Datsenko and Wanner, 2000).

Selection of positive clones is performed by colony PCR using the DreamTaq Green PCR Master Mix enzyme (Thermo Scientific™). The clone of interest is isolated on LB/agar medium (Tryptone 10 g/L, yeast extract 5 g/L, NaCl 5 g/L, Sigma-Aldrich, agar 20 g/L) containing 15 μg/mL tetracycline and incubated overnight at 37° C.

The conjugation between Escherichia coli S17-1 cells and those of strain CN0004 was adapted from the Lenz et al., 1994 protocol. The sucrose selection method (15% sucrose used) with SacB allowed precise deletion of the pta1-ackA operon. After the first homologous recombination, the clones were validated by colony PCR using the DreamTaq Green PCR Master Mix enzyme (Thermo Scientific™). After the second homologous recombination, the selection of positive clones from tetracycline-sensitive clones is performed by colony PCR using the KOD Xtreme™ Hot Start DNA Polymerase (Novagen) enzyme.

The Cupriavidus necator H16 strain Δpta1-ackA ΔphaCABΩpLAC_L-Idh C. necator is recovered and named CN0007. Genetic modifications are validated by sequencing.

Evaluation of strain CN0007 in fructose Lactate production on fructose of Cupriavidus necator strain H16 Δpta1-ackA ΔphaCABΩpLAC_L-Idh C. necator (CN0007) was evaluated using the method described in Example 3. Under these culture conditions, strain CN0007 produces 11% more lactate than strain CN0004 after 51 h of culture.

Extrapolation of CN0007 lactate production from CO₂ The lactate production of strain CN0007 was not derived from CO₂. However, the fructose evaluation showed that CN0007 produced 11% more lactate than strain CN0004 under the same culture conditions. We can extrapolate that from CO₂ as described in Example 4, strain CN0007 would produce 11% more lactate from CO₂ than strain CN0004.

### Example 10: Construction of a Genetically Modified Cupriavidus necator Strain in Which a Polyhydroxybutyrate (PHB) Biosynthetic Pathway is Deleted, an Endogenous Lactate Dehydrogenase is Overexpressed and in Which a Ferricytochrome C Reductase Lactate is Deleted (CN0008)

Strain and genetic constructs. The construction of the lactate-producing strain CN0008 is carried out according to the following protocol:

A plasmid bearing the sequences of the upstream and downstream homology zones of the active site of the L-Lactate cytochrome c reductase (IIdD, 1.1.2.3) of Cupriavidus necator is cloned in one step in vitro using the NEBuilder® HiFi DNA Assembly Cloning protocol (New England Biolabs, Evry, France). Oligonucleotides are synthesized and purified (desalted) by Eurofins Genomics.

The sequence of the upstream homology region of the gene encoding IIdD is amplified by PCR on the genomic DNA of the strain Cupriavidus necator H16 using oligonucleotides 47 (5′ CCTGCAGGTC GACTCTAGAG AGCAATTGCT CCGCCATCAG C 3′) having at the 5′ end an overlapping region of 20 base pairs with the 3′ end of plasmid pJQ200mpTet and 48 (5′ AGTCGATGGC CACTTGGCGG CGCAAGGTAC 3′) having at the 5′ end an overlapping region of 10 base pairs with the 5′ end of the downstream homology region of the gene encoding IldD and the enzyme KOD Hot Start DNA Polymerase (Novagen) with 5% DMSO. The PCR product is purified on gel with the Nucleospin gel and PCR clean-up kit (Macherey-Nagel).

The sequence of the downstream homology region of the gene encoding IldD is amplified by PCR on the genomic DNA of the Cupriavidus necator H16 strain using oligonucleotides 49 (5′ CCGCCAAGTG GCCATCGACT TGTTGCAGGC 3′) having at the 5′ end an overlapping region of 10 base pair with the 3′ end of the upstream homology of the gene encoding IIdD and 50 (5′ ATTCGAGCTC GGTACCCGGG CAAAGGCTGC GTCCAGCCAG 3′) having an overlap region of 20 base pairs with the 5′ end of the plasmid pJQ200mpTet and the enzyme KOD Hot Start DNA Polymerase (Novagen) with 5% DMSO. The PCR product is purified on gel with the Nucleospin gel and PCR clean-up kit (Macherey-Nagel).

The skeletal plasmid pJQ200mpTet is a derivative of the plasmid pJQ200mp18 (Quandt and Hynes, 1993) where the gentamicin resistance gene has been replaced by the tetracycline resistance gene. The skeletal plasmid pJQ200mpTet is cleaved by the restriction enzyme BamHI-HF (New England Biolabs, Evry, France). The digestion product is purified on gel with the Nucleospin gel and PCR clean-up kit (Macherey-Nagel).

An in vitro assembly by NEBuilder® HiFi DNA Assembly Cloning (New England Biolabs, Evry, France) is performed with the three fragments to obtain the plasmid pJQ200mpTet-ΔIIdD. 2 μL of the assembly product is transformed into chemocompetent Escherichia coli NEB 5-alpha competent cells (New England Biolabs, Evry, France). Transformants are selected on a LB/agar medium (Tryptone 10 g/L, yeast extract 5 g/L, NaCl 5 g/L, Sigma-Aldrich, agar 20 g/L) containing 15 μg/mL tetracycline. Selection of positive clones is performed by colony-based PCR using the DreamTaq Green PCR Master Mix enzyme (Thermo Scientific™). After extraction of the plasmids using the NucleoSpin® Plasmid kit (Macherey-Nagel), deletion of the IldD gene is confirmed by sequencing (Eurofins Genomics) with oligonucleotides 24 (5′ CTGGCACGAC AGGTTTCCCG A 3′), 51 (5′ TGCAAGGCGA TTAAGTTGGG TAACG 3′) and 52 (5′ GAACAGCTGC ACGCCGAG 3′).

The plasmid pJQ200mpTet -ΔIIdD is inserted into an electrocompetent Escherichia coli S17-1 strain by electroporation using a BioRad Gene Pulser electroporator (Datsenko and Wanner, 2000).

Selection of positive clones is performed by colony PCR using the DreamTaq Green PCR Master Mix enzyme (Thermo Scientific™). The clone of interest is isolated on LB/agar medium (Tryptone 10 g/L, yeast extract 5 g/L, NaCl 5 g/L, Sigma-Aldrich, agar 20 g/L) containing 15 μg/mL tetracycline and incubated overnight at 37° C.

The conjugation between Escherichia coli S17-1 cells and those of strain CN0004 was adapted from the Lenz et al., 1994 protocol. The sucrose selection method (15% sucrose used) with SacB allowed to precisely delete the gene encoding IIdD. After the first homologous recombination, the clones were validated by colony PCR using the DreamTaq Green PCR Master Mix enzyme (Thermo Scientific™). After the second homologous recombination, the selection of positive clones from tetracycline-sensitive clones is performed by colony PCR using the KOD Xtreme™ Hot Start DNA Polymerase (Novagen) enzyme.

The Cupriavidus necator H16 strain ΔIIdD ΔphaCABΩpLAC_L-Idh C. necator is recovered and named CN0008. Genetic modifications are validated by sequencing.

Evaluation of strain CN0008 in fructose Lactate production on fructose of Cupriavidus necator strain H16 ΔIIdD ΔphaCABΩpLAC_L-Idh C. necator (CN0008) was evaluated using the method described in Example 3. Under these culture conditions, strain CN0008 produces 1.4 g/L lactate after 140 h of culture.

Extrapolation of CN0008 lactate production from CO₂ The lactate production of CN0008 was not derived from CO₂. However, the fructose evaluation showed that strain CN0008 produced 7% less lactate than strain CN0004 under the same culture conditions. We can extrapolate that from CO₂ as described in Example 4, strain CN0008 would produce 7% less lactate from CO₂ than strain CN0004.

### Example 11: Construction of a Genetically Modified Cupriavidus necator Strain in Which a Polyhydroxybutyrate (PHB) Biosynthesis Pathway is Deleted, an Endogenous Lactate Dehydrogenase is Overexpressed and in Which Two Ferricytochrome C Reductase Lactates are Deleted (CN0009)

Strain and genetic constructs The construction of the lactate-producing strain CN0009 is carried out according to the following protocol:

A plasmid carrying the sequences of the upstream and downstream homology zones of the gene encoding L-lactate cytochrome reductase (IIdA) from Cupriavidus necator is cloned in one step in vitro using the NEBuilder® HiFi DNA Assembly Cloning protocol (New England Biolabs, Evry, France). Oligonucleotides are synthesized and purified (desalted) by Eurofins Genomics.

The sequence of the upstream homology region of the gene encoding IIdA is amplified by PCR on the genomic DNA of the strain Cupriavidus necator H16 using oligonucleotides 53 (5′ CCTGCAGGTC GACTCTAGAG GATCCGCAAG ACGGTTTATC TCTCGGTC 3′) having at the 5′ end an overlapping region of 25 base pairs with the 3′ end of the plasmid pJQ200mpTet and 54 (5′ GACGCTATCA CATGGGAACT CCCTTGAAAA AAACAAAAAG CTGC 3′) having at the 5′ end an overlapping region of 10 base pairs with the 5′ end of the downstream homology region of the gene encoding IIdA and the enzyme KOD Hot Start DNA Polymerase (Novagen) with 5% DMSO. The PCR product is purified on gel with the Nucleospin gel and PCR clean-up kit (Macherey-Nagel).

The sequence of the downstream homology region of the gene encoding IIdA is amplified by PCR on the genomic DNA of the Cupriavidus necator H16 strain using oligonucleotides 55 (5′ AGTTCCCATG TGATAGCGTC TATGAGGCGT C 3′) having at the 5′ end an overlapping region of 10 base pairs with the end 3′ of the upstream homology region of the gene encoding IIdA and 56 (5′ ATTCGAGCTC GGTACCCGGG GATCGAGGAA ATCGGCTGCG TAGG 3′) having at the 5′ end an overlapping region of 24 base pairs with the 5′ end of the plasmid pJQ200mpTet and the enzyme KOD Hot Start DNA Polymerase (Novagen) with 5% DMSO. The PCR product is purified on gel with the Nucleospin gel and PCR clean-up kit (Macherey-Nagel).

The skeletal plasmid pJQ200mpTet is a derivative of the plasmid pJQ200mp18 (Quandt and Hynes, 1993) where the gentamicin resistance gene has been replaced by the tetracycline resistance gene. The skeletal plasmid pJQ200mpTet is cleaved by the restriction enzyme BamHl-HF (New England Biolabs, Evry, France). The digestion product is purified on gel with the Nucleospin gel and PCR clean-up kit (Macherey-Nagel).

An in vitro assembly by NEBuilder® HiFi DNA Assembly Cloning (New England Biolabs, Evry, France) is performed with the three fragments to obtain the plasmid pJQ200mpTet-ΔIIdA. 2 μL of the assembly product is transformed into competent chemocompetent Escherichia coli NEB 5-alpha cells (New England Biolabs, Evry, France). Transformants are selected on a LB/agar medium (Tryptone 10 g/L, yeast extract 5 g/L, NaCl 5 g/L, Sigma-Aldrich, agar 20 g/L) containing 15 μg/mL tetracycline. Selection of positive clones is performed by colony PCR using the DreamTaq Green PCR Master Mix enzyme (Thermo Scientific™). After extraction of the plasmids using the NucleoSpin® Plasmid kit (Macherey-Nagel), deletion of the IIdA gene is confirmed by sequencing (Eurofins Genomics) with oligonucleotides 21 (5′ TGCAAGGCGA TTAAGTTG 3′), 57 (5′ CCTCATAGAC GCTATCACAT GG 3′), 58 (5′ CCATGTGATAGCGTCTATGAGG 3′) and 24 (5′ CTGGCACGACAGGTTTCCCGA 3′).

The plasmid pJQ200mpTet-ΔIIdA is inserted into an electrocompetent Escherichia coli S17-1 strain by electroporation using a BioRad Gene Pulser electroporator (Datsenko and Wanner, 2000).

Selection of positive clones is performed by colony PCR using the DreamTaq Green PCR Master Mix enzyme (Thermo Scientific™). The clone of interest is isolated on LB/agar medium (Tryptone 10 g/L, yeast extract 5 g/L, NaCl 5 g/L, Sigma-Aldrich, agar 20 g/L) containing 15 μg/mL tetracycline and incubated overnight at 37° C.

The conjugation between Escherichia coli S17-1 cells and those of strain CN0008 was adapted from the Lenz et al., 1994 protocol. The sucrose selection method (15% sucrose used) with SacB allowed to precisely delete the gene encoding IIdA. After the first homologous recombination, the clones were validated by colony PCR using the DreamTaq Green PCR Master Mix enzyme (Thermo Scientific™). After the second homologous recombination, selection of positive clones from tetracycline-sensitive clones is performed by colony PCR using the KOD Xtreme™ Hot Start DNA Polymerase (Novagen) enzyme.

The strain Cupriavidus necator H16 ΔIIdA ΔIIdD ΔphaCABΩpLAC_L-Idh C. necator is recovered and named CN0009. Genetic modifications are validated by sequencing.

Evaluation of strain CN0009 for fructose Lactate production on fructose of Cupriavidus necator strain H16 ΔIIdD ΔphaCABΩpLAC_L-Idh C. necator (CN0009) was evaluated using the method described in Example 3. Under these culture conditions, strain CN0009 produces 1.4 g/L lactate after 140 h of culture.

Extrapolation of CN0009 lactate production from CO₂ The lactate production of strain CN0009 was not derived from CO₂. However, the fructose evaluation showed that strain CN0009 produced 7% less lactate than strain CN0004 under the same culture conditions. We can extrapolate that from CO₂ as described in Example 4, strain CN0009 would produce 7% less lactate from CO₂ than strain CN0004.

### Example 12: Construction of a Genetically Modified Cupriavidus necator Strain in Which a Polyhydroxybutyrate (PHB) Biosynthetic Pathway is Deleted, an Endogenous Lactate Dehydrogenase is Overexpressed and in Which a Phosphoenolpyruvate Synthase is Deleted (CNO010)

Strain and genetic constructs. The construction of the lactate-producing strain CN0010 is carried out according to the following protocol:

A plasmid carrying the sequences of the upstream and downstream homology zones of the gene encoding Cupriavidus necator phosphoenolpyruvate synthase (ppsA) is cloned in one step in vitro using the NEBuilder® HiFi DNA Assembly Cloning protocol (New England Biolabs, Evry, France). Oligonucleotides are synthesized and purified (desalted) by Eurofins Genomics.

The sequence of the upstream homology region of the gene encoding ppsA is amplified by PCR on the genomic DNA of the Cupriavidus necator H16 strain using oligonucleotides 59 (5′ AGATCCTTTA ATTCGAGCTC GGTACCCGAA GATCTTCGGC TTGAACG 3′) having at the 5′ end an overlapping region of 25 base pairs with the 3′ end of the plasmid pLO3 and 60 (5′ ACGTCAAATG CTTCACATGT CCGGTATGTT CTTGGAGTTC 3′) having at the 5′ end an overlapping region of 15 base pairs with the 5′ end of the downstream homology region of the gene encoding ppsA and the enzyme KOD Hot Start DNA Polymerase (Novagen) with 5% DMSO. The PCR product is purified on gel with the Nucleospin gel and PCR clean-up kit (Macherey-Nagel).

The sequence of the downstream homology region of the gene encoding ppsA is amplified by PCR on the genomic DNA of the Cupriavidus necator H16 strain using oligonucleotides 61 (5′ AACATACCGG ACATGTGAAG CATTTGACGT CACAATAACG 3′) having at the 5′ end an overlapping region of 15 base pairs with the 3′ end of the upstream homology zone of the gene encoding ppsA and 62 (5′ ACTTAATTAA GGATCCGGCG CGCCCCTTGA GCACGTGCT TGTAGG 3′) having at the 5′ end an overlapping zone of 25 base pairs with the 5′ end of plasmid pLO3 and the enzyme KOD Hot Start DNA Polymerase (Novagen) with 5% DMSO. The PCR product is purified on gel with the Nucleospin gel and PCR clean-up kit (Macherey-Nagel).

The skeletal plasmid pLO3 (Lenz and Friedrich, 1998) is cleaved by the restriction enzyme Xmal-HF (New England Biolabs, Evry, France). The digestion product is purified on gel with the Nucleospin gel and PCR clean-up kit (Macherey-Nagel).

An in vitro assembly by NEBuilder® HiFi DNA Assembly Cloning (New England Biolabs, Evry, France) is performed with the three fragments to obtain the plasmid pLO3-ΔppsA. 2 μL of the assembly product is transformed into chemocompetent, NEB 5-alpha competent Escherichia coli cells (New England Biolabs, Evry, France) cells. Transformants are selected on a LB/agar medium (Tryptone 10 g/L, yeast extract 5 g/L, NaCl 5 g/L, Sigma-Aldrich, agar 20 g/L) containing 15 μg/mL tetracycline. Selection of positive clones is performed by colony PCR using the DreamTaq Green PCR Master Mix enzyme (Thermo Scientific™). After extraction of the plasmids using the NucleoSpin® Plasmid kit (Macherey-Nagel), deletion of the ppsA gene is confirmed by sequencing (Eurofins Genomics) with oligonucleotides 63 (5′ ATGAACACCG GCACCTTCTA CC 3′), 91 (5′ CAGGATGGAG TGGCTGAACG 3′) and 29 (5′ GCAAACAAAC CACCGCTGGT 3′).

The plasmid pLO3-ΔppsA is inserted into an electrocompetent Escherichia coli S17-1 strain by electroporation using a BioRad Gene Pulser electroporator (Datsenko and Wanner, 2000).

Selection of positive clones is performed by colony PCR using the DreamTaq Green PCR Master Mix enzyme (Thermo Scientific™). The clone of interest is isolated on LB/agar medium (Tryptone 10 g/L, yeast extract 5 g/L, NaCl 5 g/L, Sigma-Aldrich, agar 20 g/L) containing 15 μg/mL tetracycline and incubated overnight at 37° C.

The conjugation between Escherichia coli S17-1 cells and those of strain CN0004 was adapted from the Lenz et al., 1994 protocol. The sucrose selection method (15% sucrose used) with SacB allowed to precisely delete the gene encoding ppsA. After the first homologous recombination, the clones were validated by colony PCR using the DreamTaq Green PCR Master Mix enzyme (Thermo Scientific™). After the second homologous recombination, the selection of positive clones from tetracycline-sensitive clones is performed by colony PCR using the KOD Xtreme™ Hot Start DNA Polymerase (Novagen) enzyme.

The Cupriavidus necator strain H16 ΔppsA ΔphaCABΩpLAC_L-Idh C. necator is recovered and named CN0010. Genetic modifications are validated by sequencing.

### REFERENCES

Angermayr & al., 2012. Appl. Environ. Microbiol., 78, 19, 7098-7106
Barnard et al. 2015—Appl. Environ. Microbiol. 71, 5735-5742.
Bi & al., 2013. Microb. Cell Fact. 12, 1-10.

### Daboussi et al., Nucleic Acids Res. 2012. 40: 6367-79

### Daneholt, 2006. Nobel Prize in Physiology or Medicine

Datsenko & al. Proc. Natl. Acad. Sci. U.S.A. 97, 6640-5

### Grousseau et al., 2014—Applied Microbiology and Biotechnology, 98(9)

### Gruber et al., 2014—J. Biotechnol. 186, 74-82

### Kaufmann et al., Methods Mol Biol. 2011; 765: 275-94.

### Kim et al., PNAS; 93: 1156-1160

Krauβe & al., 2009. J. Mol. Microbiol. Biotechnol. 17, 146-152
Lenz & Friedrich, 1998. Proc. Natl. Acad. Sci. 95, 12474-12479

### Lenz & al., 1994. J. Bacteriol. 176, 4385-93

### Lodish et al., Molecular Cell Biology 4th ed. 2000

Lütte et al., 2012—Appl. Environ. Microbiol. 78, 7884-7890)

### Mali et al., Nat Methods. 2013 Oct; 10(10): 957-63.

### Ousterout et al., Methods Mol Biol. 2016; 1338: 27-42.

### Quandt & Hynes, 1993. Gene 127, 15-21

Schlegel & Vollbrecht, 1980. J. Gen. Microbiol. 117, 475-481.

### Sydow et al., 2017—Journal of Biotechnology. 263, 1-10

### Thomas et al., Cell. 1987; 51: 503-12

### WO 2014/205146, WO 2015/155790

1. A naturally hydrogen-oxidizing bacterium which is genetically modified to produce lactate from CO₂, wherein said bacterium is genetically modified to overexpress at least one gene encoding a lactate dehydrogenase.

2. The bacterium according to claim 1, wherein said overexpressed at least one gene encoding a lactate dehydrogenase is at least one gene encoding an endogenous lactate dehydrogenase.

3. The bacterium according to claim 1, wherein said overexpressed at least one gene encoding a lactate dehydrogenase is at least one gene encoding an exogenous lactate dehydrogenase, preferentially derived from a bacterium, fungus, yeast or mammal, more preferentially derived from a bacterium.

4. The bacterium according to claim 1, wherein said bacterium is genetically modified to overexpress at least one gene encoding an L-lactate dehydrogenase and optionally wherein the expression of at least one gene encoding a D-lactate dehydrogenase is at least partially inhibited.

5. The bacterium according to claim 1, wherein said bacterium is genetically modified to overexpress at least one gene encoding a D-lactate dehydrogenase and optionally wherein the expression of at least one gene encoding an L-lactate dehydrogenase is at least partially inhibited.

6. The bacterium according to claim 4, wherein said bacterium is further genetically modified to at least partially inhibit at least one pyruvate degradation pathway competing with the lactate synthesis pathway.

7. The bacterium according to claim 6, wherein said at least one pyruvate degradation pathway competing with the lactate synthesis pathway at least partially inhibited is the pathway for the synthesis of polyhydroxybutyrate (PHB).

8. The bacterium according to claim 6, wherein said at least one pyruvate degradation pathway competing with the lactate synthesis pathway at least partially inhibited comprises at least partial inhibition of the expression of at least one gene selected from the genes encoding acetyl-CoA acetyltransferase, acetoacetyl-CoA reductase and poly(3-hydroxybutyrate) synthase is at least partially inhibited.

9. The bacterium according to claim 6, wherein said at least one pyruvate degradation pathway competing with the lactate synthesis pathway at least partially inhibited comprises at least partial inhibition of the expression of at least one gene selected from genes encoding a phosphoenolpyruvate synthase and a pyruvate carboxylase.

10. The bacterium according to claim 6, wherein said at least one pyruvate degradation pathway competing with the lactate synthesis pathway at least partially inhibited comprises at least partial inhibition of the route of conversion of acetyl-CoA to acetate and/or acetaldehyde.

11. The bacterium according to claim 1, wherein the expression of at least one gene encoding a lactate ferricytochrome C reductase is at least partially inhibited.

12. (canceled)

13. A process for producing lactate from CO₂, comprising the steps consisting in
culturing a naturally hydrogen-oxidizing bacterium which is genetically modified to produce lactate from CO₂, wherein said bacterium is genetically modified to overexpress at least one gene encoding a lactate dehydrogenase in the presence of CO₂ as the sole source of carbon, then
recovering the lactate from the fermentation medium.

14. The bacterium according to claim 3, wherein said at least one gene encoding an exogenous lactate dehydrogenase is derived from a bacterium, fungus, yeast or mammal.

15. The bacterium according to claim 3, wherein said at least one gene encoding an exogenous lactate dehydrogenase is derived from a bacterium.

16. The bacterium according to claim 6, wherein said at least one pyruvate degradation pathway competing with the lactate synthesis pathway at least partially inhibited comprises at least partial inhibition of the expression of two or three of the genes selected from the genes encoding acetyl-CoA acetyltransferase, acetoacetyl-CoA reductase and poly(3-hydroxybutyrate) synthase.

17. The bacterium according to claim 6, wherein said at least one pyruvate degradation pathway competing with the lactate synthesis pathway at least partially inhibited comprises at least partial inhibition of the expression of at least one gene selected from genes encoding a phosphate acetyltransferase, an acetate kinase and an acetaldehyde dehydrogenase.

18. The bacterium according to claim 1, wherein it is selected from the group consisting of Ralstonia sp., Cupriavidus sp., Hydrogenobacter sp., Rhodococcus sp., Hydrogenovibrio sp.; Rhodopseudomonas sp., Rhodobacter sp, Aquifex sp., Cupriavidus sp., Couynebacterium sp., Nocardia sp., Rhodopseudomonas sp., Rhodospirillum sp., Rhodococcus sp., Rhizobium sp., Thiocapsa sp., Pseudomonas sp., Hydrogenomonas sp., Hydrogenobacter sp., Hydrogenophilus sp., Hydrogenautresmus sp., Helicobacter sp., Xanthobacter sp., Hydrogenophaga sp., Bradyrhizobium sp., Alcaligenes sp., Amycolata sp., Aquaspirillum sp., Arthrobacter sp., Azospirillum sp., Variovouax sp., Acidovouax sp., Bacillus sp., Calderobacterium sp., Derxia sp., Flavobacterium sp., Microcyclus sp., Mycobacterium sp., Paracoccus sp., Persephonella sp., Renobacter sp., Thermocrinis sp., Wautersia sp., and cyanobacteria.

19. The bacterium of claim 1, wherein said bacterium further comprises the following genetic modifications:
at least partial inhibition of the pathway for the synthesis of polyhydroxybutyrate (PHB)
at least partial inhibition of the expression of at least one gene selected from genes encoding a phosphoenolpyruvate synthase and a pyruvate carboxylase
at least partial inhibition of the route of conversion of acetyl-CoA to acetate and/or acetaldehyde, and
at least partial inhibition of at least one gene encoding a lactate ferricytochrome C reductase.


SEQUENCE LISTING (87 sequences)

SEQ ID NO: 1 - protein, 349 aa, Cupriavidus necator H16
MKISLTSARQLARDILAAQQVPADIADDVAEHLVESDRCGYISHGLSILPNYRTALDGHSVNPQGRAKCVLDQGTLMVFDGDGGFGQHVGKSVMQAAIERVRQHGHCIVTLRRSHHLGRMGHYGEMAAAAGFVLLSFTNVINRAPVVAPFGGRVARLTTNPLCFAGPMPNGRPPLVVDIATSAIAINKARVLAEKGEPAPEGSIIGADGNPTTDASTMFGEHPGALLPFGGHKGYALGVVAELLAGVLSGGGTIQPDNPRGGVATNNLFAVLLNPALDLGLDWQSAEVEAFVRYLHDTPPAPGVDRVQYPGEYEAANRAQASDTLNINPAIWRNLERLAQSLNVAVPTA

SEQ ID NO: 2 - protein, 323 aa, Pediococcus acidilactici
MSNIQNHQKVVLVGDGAVGSSYAFAMAQQGIAEEFVIVDVVKDRTVGDALDLEDATPFTAPKNIYSGEYSDCKDADLVVITAGAPQKPGETRLDLVNKNLNILSTIVKPVVDSGFDGIFLVAANPVDILTYATWKFSGFPKEKVIGSGISLDTARLRVALGKKFNVSPESVDAYILGEHGDSEFAAYSSATIGTKPLLEIAKEEGVSTDELAEIEDSVRNKAYEIINKKGATFYGVGTALMRISKAILRDENAVLPVGAYMDGEYGLNDIYIGTPAVINGQGLNRVIEAPLSDDEKKKMTDSATTLKKVLTDGLNALAEKQDK

SEQ ID NO: 3 - protein, 329 aa, Streptococcus equinus
MTATKQHKKVILVGDGAVGSSYAFALVNQGIAQELGIIEIPQLFNKAVGDAEDLSHALAFTSPKKIYAAKYEDCADADLVVITAGAPQKPGETRLDLVGKNLAINKSIVTEVVKSGFKGIFLVAANPVDVLTYSTWKFSGFPKERVIGSGTSLDSARFRQALAEKLDVDARSVHAYIMGEHGDSEFAVWSHANVAGVNLESYLKDVQNVDEAELVELFEGVRDAAYSIINKKGATFYGIAVALARITKAILNDENAVLPLSVFQEGQYANVTDCYIGQPAIVGAHGIVRPVNIPLNDAEQQKMEASAKELKAIIDEAFSKEEFASACKN

SEQ ID NO: 4 - protein, 312 aa, Bacillus coagulans
MKKVNRIAVVGTGAVGTSYCYAMINQGVAEELVLIDINEAKAEGEAMDLNHGLPFAPTSTRVWKGDYSDCGTADLVVITAGSPQKPGETRLDLVAKNAKIFKGMIKSIMDSGFNGIFLVASNPVDILTYVTWKESGLPKEHVIGSGTVLDSARLRNSLSAQFGIDPRNVHAAIIGEHGDTELPVWSHTNIGYDTIESYLQKGIIDEKTLDDIFVNTRDAAYHIIERKGATFYGIGMSLTRITRAILNNENSVLTVSAFLEGQYGNSDVYVGVPAIINRQGIREVVEIKLNEKEQEQFNHSVKVLKETMAPVL

SEQ ID NO: 5 - protein, 312 aa, Lactobacillus casei
MRNNGNIILIGDGAIGSSYAFNCLTTGVGQSLGIIDVNEKRVQGDVEDLSDSLPYTSQKNIYAASYEDCKYADIIVITAGIAQKPGQTRLQLLAINAKIMKEITHNIMASGFNGFILVASNPVDVLAELVLQESGLPRNQVLGSGTALDSARLRSEIGLRYNVDARIVHGYIMGEHGDSEFPVWDYTNIGGKPILDWIPKDRQDKDLPDISERVKTAAYGIIEKKGATFYGIAASLTRLTSAFLNDDRAAFAMSVHLEGEYGLSGVSIGVPVILGANGLERIIELDLNPEDHKRLADSAAILKENLKKAQEA

SEQ ID NO: 6 - protein, 323 aa, Lactobacillus helveticus
MAREEKPRKVILVGDGAVGSTFAFSMVQQGIAEELGIIDIAKEHVEGDAIDLADATPWTSPKNIYAADYPDCKDADLVVITAGAPQKPGETRLDLVNKNLKILSSIVEPVVESGFEGIFLVVANPVDILTHATWRMSGFPKDRVIGSGTSLDTGRLQKVIGKMENVDPSSVNAYMLGEHGDTEFPAWSYNNVAGVKVADWVKAHNMPESKLEDIHQEVKDMAYDIINKKGATFYGIGTASAMIAKAILNDEHRVLPLSVPMDGEYGLHDLHIGTPAVVGRKGLEQVIEMPLSDKEQELMTASADQLKKVMDKAFKETGVKVRQ

SEQ ID NO: 7 - protein, 302 aa, Lactobacillus_bulgaricus
MRKVAVIGMGHVGATAAFILFTHGVADELVLLDKNETKCRAEWGDLRDTLGRNDFYVNVKWGDWKELADADLIITAFGDVAASITTGDRFAEFPINTKNAVEVGQKIKDSGFKGVIINISNPCDVVTSILQKVTGLPKSQVFGTGTFLDTSRMQRVVGEKLGQDPRNVSGFNLGEHGSSQFTAWSTVWVNNRPAKDLFNEAEKEEMDRLSKDNAFMVGKGKGYTCYAVATCAVRLARAVFSDAKFYGPTSCYVESLGTYIGYPSIVGKHGVEEVPVLDLPADEQAKLEASAKKLKDSLASLE

SEQ ID NO: 8 - protein, 320 aa, Lactobacillus_plantarum
MSSMPNHQKVVLVGDGAVGSSYAFAMAQQGIAEEFVIVDVVKDRTKGDALDLEDAQAFTAPKKIYSGEYSDCKDADLVVITAGAPQKPGESRLDLVNKNLNILSSIVKPVVDSGFDGIFLVAANPVDILTYATWKFSGFPKDRVIGSGTSLDSSRLRVALGKQFNVDPRSVDAYIMGEHGDSEFAAYSTATIGTRPVRDVAKEQGVSDEDLAKLEDGVRNKAYDIINLKGATFYGIGTALMRISKAILRDENAVLPVGAYMDGQYGLNDIYIGTPAVIGGTGLKQIIESPLSADELKKMQDSAATLKKVLNDGLAELENK

SEQ ID NO: 9 - protein, 320 aa, Lactobacillus_pentosus
MSSMPNHQKVVLVGDGAVGSSYAFAMAQQGIAEEFVIVDVVKDRTKGDALDLEDAQAFTAPKKIYSGEYSDCKDADLVVITAGAPQKPGESRLDLVNKNLNILSSIVKPVVDSGFDGIFLVAANPVDILTYATWKFSGFPKDRVIGSGTSLDTSRLRVALGKQFNVDPRSVDAYIMGEHGDSEFAAYSTATIGTRPVRDVAKEQGVSDDDLAKLEDGVRNKAYDIINLKGATFYGIGTALMRISKAILRDENAILPVGAYMDGQYGLNDIYIGTPAVIGGTGLKQIIESPLSADELKKMQDSAATLKKVLNDGLAELENK

SEQ ID NO: 10 - protein, 323 aa, Lactococcus_lactis
MKINNKKVVIVGAGAVGSTYAHNLVVDDLADEIAIINTNKSKASANSLDLLHALPYLNAAPKNIYAADYSDVSDADIVVLSANAPSATFGKNPDRLQLLENNVEMIRDITRKTMDAGFDGIFLVASNPVDVLAQVVAEVSGLPKHRVIGTGTLLETSRMRQIVAEKLQINPKSIHGYVLAEHGKSSFAAWSNVTVGAIPLTTWLKKYPNPEFPTFDEIDQEIREVGLDIFMQKGNTSYGIAASLARLTRAIFRNESVILPVSAYLTGEYGQSNLYTGSPAIIDRTGVRAVLELELTQDEQEKFKASTVLLKENFDSIREKCTL

SEQ ID NO: 11 - protein, 331 aa, Cupriavidus necator_H16
MEIAVFSAKSYDRQHLDAANAAEGHQLKYFEVPLDNETVGLAAGHGAVCIFVNDRADATVLEALGRGGTKLVALRCTGFNNVDLKAAQALGIKVVRVVDYSPNAVAEHAAALLMAVNRKIHRAYNRTRDFNFSLEGLMGFDLCGKTVAVIGTGKIGRVFAKIMVGFGCNVIGYDKYPSPEFEALGGRYADEGEIGASADCISLHCPLTPETHHIINAETLSRAKPGALLINTSRGGLIDTEAVIGALRSGQLGGLAIDVYEQEAGLFFRDLSGIIVDDSVLQQLITFPNVIVTGHQAFLTREAVTTICETTLRSVTEFESGKPLTNEVGAG

SEQ ID NO: 12 - protein, 333 aa, Lactobacillus_delbrueckii_subsp_bulgaricus
MTKIFAYAIREDEKPFLKEWEDAHKDVEVEYTDKLLTPETVALAKGADGVVVYQQLDYTAETLQALADNGITKMSLRNVGVDNIDMAKAKELGFQITNVPVYSPNAIAEHAAIQAARILRQDKAMDEKVARHDLRWAPTIGREVRDQVVGVIGTGHIGQVFMQIMEGFGAKVIAYDIFRNPELEKKGYYVDSLDDLYKQADVISLHVPDVPANVHMINDESIAKMKQDVVIVNVSRGPLVDTDAVIRGLDSGKIFGYAMDVYEGEVGIFNEDWEGKEFPDARLADLIARPNVLVTPHTAFYTTHAVRNMVVKAFDNNLELVEGKEAETPVKVG

SEQ ID NO: 13 - protein, 329 aa, Escherichia_coli_K-12
MKLAVYSTKQYDKKYLQQVNESFGFELEFFDFLLTEKTAKTANGCEAVCIFVNDDGSRPVLEELKKHGVKYIALRCAGFNNVDLDAAKELGLKVVRVPAYDPEAVAEHAIGMMMTLNRRIHRAYQRTRDANFSLEGLTGFTMYGKTAGVIGTGKIGVAMLRILKGFGMRLLAFDPYPSAAALELGVEYVDLPTLFSESDVISLHCPLTPENYHLLNEAAFEQMKNGVMIVNTSRGALIDSQAAIEALKNQKIGSLGMDVYENERDLFFEDKSNDVIQDDVFRRLSACHNVLFTGHQAFLTAEALTSISQTTLQNLSNLEKGETCPNELV

SEQ ID NO: 14 - protein, 329 aa, Bacillus_coagulans
MRKVVAYETRADEFPLFQKFARKFDLDIKYVDDVLTPQTAVEAKGAEAVTILGNYPVGAETFMALRDASVKYIGLRTAGYNHIDQEAAKAYGIRFSNVAYSPYCVADFATMLILMCVRKAKQILSRVEAQDFSVEGIQGREMHNLTIGIIGAGRIGSIVAKNLSGFGCNIIAHDTVERDELRGILKYVSLDELLKESDVITIHTPLFDRTYHMINQDRIAKMKDGVCIINCSRGAVVDTDALIAGIEAGKVGAAGIDVLEDEEGIFHYDRRTDILAHHQLAILRSFPNVIVTPHTAFYTDQAVSDMVEMALTSLVSFMETGKSRWEIKS

SEQ ID NO: 15 - DNA, 1050 bp, Ralstonia eutropha H16
tcaggccgtggggacggccacgttgagcgactgcgccaggcgctcaagattgcgccagatggccgggttgatgtttagcgtgtcgctggcctgcgcccggttggcggcctcgtactcgccggggtactgcacgcggtcgacgcccggcgccggcggtgtgtcgtgcaggtagcgcacgaacgcctcgacctcggcgctctgccagtccaggcccaggtccagcgcgggattgagcagcaccgcgaacaggttgttggtggccacgccgccgcgcggattgtctggctggatggtaccgccgccggacagcacgcccgccagcagctcggccacaacgcccagtgcgtagcccttgtggccgccaaagggcagcagcgcgccggggtgttcgccgaacatggttgacgcgtcggtggtggggttgccgtcggcgccgatgatgctgccttcgggcgccggctcgcctttctcggccagcacacgggccttgttgatggcaatcgcgctggtggcgatgtccaccaccagaggcggccgcccgttgggcatcgggccggcgaaacacagcgggttggtggtgagccgcgccacgcggccgccgaacggcgccaccaccggcgcgcggttgatcacgttggtgaagctcagcagcacaaagccggcggcggccgccatctcgccgtagtggcccatgcggccgagatggtgcgagcggcgcagagtgacgatgcagtggccatgctggcgcacgcgctcgatcgctgcttgcatcacggacttgcccacgtgctggccgaagccgccgtcgccgtcgaacaccatcagcgtgccctggtccagcacgcatttggcgcggccttgcgggttgacgctgtggccgtcgagggcggtgcggtagttgggcaggatcgacaggccgtggctgatatagccgcagcggtcggattcgaccaggtgctcggccacgtcgtcagcgatgtcggcgggcacctgctgcgcggcgaggatgtcgcgggcaagctggcgggcgctggtgagggagatcttcat

SEQ ID NO: 16 - DNA, 26 bp, artificial sequence
  oligonucleotide
ggatccaaactcgagtaaggatctcc

SEQ ID NO: 17 - DNA, 36 bp, artificial sequence
  oligonucleotide
atgtatatctccttcttaaaagatcttttgaattcc

SEQ ID NO: 18 - DNA, 37 bp, ARTIFICIAL SEQUENCE
  OLIGONUCLEOTIDE
gaaggagatatacatatgaagatctccctcaccagcg

SEQ ID NO: 19 - DNA, 33 bp, ARTIFICIAL SEQUENCE
  OLIGONUCLEOTIDE
ctcgagtttggatcctcaggccgtggggacggc

SEQ ID NO: 20 - DNA, 27 bp, ARTIFICIAL SEQUENCE
  OLIGONUCLEOTIDE
gacgctttttatcgcaactctctactg

SEQ ID NO: 21 - DNA, 24 bp, ARTIFICIAL SEQUENCE
  OLIGONUCLEOTIDE
cgaacgccctaggtataaacgcag

SEQ ID NO: 22 - DNA, 36 bp, Artificial Sequence
  Oligonucleotide
aaggagatatacatatgaagatctccctcaccagcg

SEQ ID NO: 23 - DNA, 34 bp, Artificial Sequence
  Oligonucleotide
actcgagtttggatcctcaggccgtggggacggc

SEQ ID NO: 24 - DNA, 22 bp, Artificial Sequence
  Oligonucleotide
cgaaggtgagccagtgtgactc

SEQ ID NO: 25 - DNA, 22 bp, Artificial Sequence
  Oligonucleotide
cctgtcgatcctgcccaactac

SEQ ID NO: 26 - DNA, 24 bp, Artificial Sequence
  Oligonucleotide
cattgattatttgcacggcgtcac

SEQ ID NO: 27 - DNA, 35 bp, Artificial Sequence
  Oligonucleotide
aaggagatatacatatgaccgcgaccaagcagcac

SEQ ID NO: 28 - DNA, 39 bp, Artificial Sequence
  Oligonucleotide
actcgagtttggatcctcagttcttgcaggccgacgcga

SEQ ID NO: 29 - DNA, 22 bp, Artificial Sequence
  Oligonucleotide
ggagctgggcatcatcgagatc

SEQ ID NO: 30 - DNA, 112 bp, Artificial Sequence
  Oligonucleotide
agacaatcaaatctttacactttatgcttccggctcgtatgttgtgtggaattgtgagcggataacaatttcacacaggaaacagctatgaagatctccctcaccagcgccc

SEQ ID NO: 31 - DNA, 33 bp, Artificial Sequence
  Oligonucleotide
ccaggccggcaggtcaggccgtggggacggcca

SEQ ID NO: 32 - DNA, 45 bp, Artificial Sequence
  Oligonucleotide
gcatgcctgcaggtcgactctagagggtcgcttctactcctatcg

SEQ ID NO: 33 - DNA, 33 bp, Artificial Sequence
  Oligonucleotide
cataaagtgtaaagatttgattgtctctctgcc

SEQ ID NO: 34 - DNA, 33 bp, Artificial Sequence
  Oligonucleotide
ccccacggcctgacctgccggcctggttcaacc

SEQ ID NO: 35 - DNA, 48 bp, Artificial Sequence
  Oligonucleotide
tacgaattcgagctcggtacccgggttctggatgtcgatgaaggcctg

SEQ ID NO: 36 - DNA, 18 bp, Artificial Sequence
  Oligonucleotide
tgcaaggcgattaagttg

SEQ ID NO: 37 - DNA, 20 bp, Artificial Sequence
  Oligonucleotide
catgcaaagtgccggccagg

SEQ ID NO: 38 - DNA, 22 bp, Artificial Sequence
  Oligonucleotide
ctgcacgaacatggtgctggct

SEQ ID NO: 39 - DNA, 21 bp, Artificial Sequence
  Oligonucleotide
ctggcacgacaggtttcccga

SEQ ID NO: 40 - DNA, 46 bp, Artificial Sequence
  Oligonucleotide
agatcctttaattcgagctcggtaccgcatggccaaggtggaagag

SEQ ID NO: 41 - DNA, 37 bp, Artificial Sequence
  Oligonucleotide
tgccggccaacgtcacatgggatgcagggaagcgaac

SEQ ID NO: 42 - DNA, 33 bp, Artificial Sequence
  Oligonucleotide
tccctgcatcccatgtgacgttggccggcaggg

SEQ ID NO: 43 - DNA, 57 bp, Artificial Sequence
  Oligonucleotide
acttaattaaggatccggcgcgccccccgggctgatagttcttcaacaccagcagtc

SEQ ID NO: 44 - DNA, 20 bp, Artificial Sequence
  oligonucleotide
gcaaacaaaccaccgctggt

SEQ ID NO: 45 - DNA, 20 bp, Artificial Sequence
  Oligonucleotide
cgccatatcggatgccgttc

SEQ ID NO: 46 - DNA, 22 bp, Artificial Sequence
  Oligonucleotide
tagcagcacgccatagtgactg

SEQ ID NO: 47 - DNA, 45 bp, Artificial Sequence
  Oligonucleotide
tcctttaattcgagctcggtacccgggtgcgtaatccacttccag

SEQ ID NO: 48 - DNA, 37 bp, Artificial Sequence
  Oligonucleotide
cccatcgttcacacggcaagtctccgttaaggaattc

SEQ ID NO: 49 - DNA, 32 bp, Artificial Sequence
  Oligonucleotide
gacttgccgtgtgaacgatgggccatcgggca

SEQ ID NO: 50 - DNA, 45 bp, Artificial Sequence
  Oligonucleotide
taaggatccggcgcgcccccgggttgagcaggatcacgtcgatcc

SEQ ID NO: 51 - DNA, 45 bp, Artificial Sequence; identical to SEQ ID NO: 47
  Oligonucleotide

SEQ ID NO: 52 - DNA, 37 bp, Artificial Sequence; identical to SEQ ID NO: 48
  Oligonucleotide

SEQ ID NO: 53 - DNA, 32 bp, Artificial Sequence; identical to SEQ ID NO: 49
  Oligonucleotide

SEQ ID NO: 54 - DNA, 45 bp, Artificial Sequence; identical to SEQ ID NO: 50
  Oligonucleotide

SEQ ID NO: 55 - DNA, 23 bp, Artificial Sequence
  Oligonucleotide
taatccacttccagcgcgataag

SEQ ID NO: 56 - DNA, 22 bp, Artificial Sequence
  oligonucleotide
gttcgaagccaccgagtatgac

SEQ ID NO: 57 - DNA, 20 bp, Artificial Sequence
  Oligonucleotide
gcaaacaaaccaccgctggt

SEQ ID NO: 58 - DNA, 45 bp, Artificial Sequence
  Oligonucleotide
tcctttaattcgagctcggtacgtgtccaatgagatgacagcacg

SEQ ID NO: 59 - DNA, 30 bp, Artificial Sequence
  oligonucleotide
tgtagcggtggtgcgtcagggtcgtcggtg

SEQ ID NO: 60 - DNA, 32 bp, Artificial Sequence
  Oligonucleotide
accctgacgcaccaccgctacagccgaccaag

SEQ ID NO: 61 - DNA, 47 bp, Artificial Sequence
  Oligonucleotide
taaggatccggcgcgcccccgggctgatacgttcacgcatagtggtc

SEQ ID NO: 62 - DNA, 19 bp, Artificial Sequence
  Oligonucleotide
gacttccggcaggtcatgc

SEQ ID NO: 63 - DNA, 21 bp, Artificial Sequence
  Oligonucleotide
cagttgttgcgctgcagtcat

SEQ ID NO: 64 - DNA, 18 bp, Artificial Sequence
  Oligonucleotide
gccaagccggaacgcgtc

SEQ ID NO: 65 - DNA, 20 bp, Artificial Sequence
  Oligonucleotide
gatggtggcacgatgttcac

SEQ ID NO: 66 - DNA, 41 bp, Artificial Sequence
  Oligonucleotide
cctgcaggtcgactctagagagcaattgctccgccatcagc

SEQ ID NO: 67 - DNA, 30 bp, Artificial Sequence
  Oligonucleotide
agtcgatggccacttggcggcgcaaggtac

SEQ ID NO: 68 - DNA, 30 bp, Artificial Sequence
  Oligonucleotide
ccgccaagtggccatcgacttgttgcaggc

SEQ ID NO: 69 - DNA, 40 bp, Artificial Sequence
  oligonucleotide
attcgagctcggtacccgggcaaaggctgcgtccagccag

SEQ ID NO: 70 - DNA, 21 bp, Artificial Sequence
  Oligonucleotide
ctggcacgacaggtttcccga

SEQ ID NO: 71 - DNA, 25 bp, Artificial Sequence
  oligonucleotide
tgcaaggcgattaagttgggtaacg

SEQ ID NO: 72 - DNA, 18 bp, Artificial Sequence
  oligonucleotide
gaacagctgcacgccgag

SEQ ID NO: 73 - DNA, 48 bp, Artificial Sequence
  Oligonucleotide
cctgcaggtcgactctagaggatccgcaagacggtttatctctcggtc

SEQ ID NO: 74 - DNA, 44 bp, Artificial Sequence
  Oligonucleotide
gacgctatcacatgggaactcccttgaaaaaaacaaaaagctgc

SEQ ID NO: 75 - DNA, 31 bp, Artificial Sequence
  Oligonucleotide
agttcccatgtgatagcgtctatgaggcgtc

SEQ ID NO: 76 - DNA, 44 bp, Artificial Sequence
  Oligonucleotide
attcgagctcggtacccggggatcgaggaaatcggctgcgtagg

SEQ ID NO: 77 - DNA, 18 bp, Artificial Sequence
  Oligonucleotide
tgcaaggcgattaagttg

SEQ ID NO: 78 - DNA, 22 bp, Artificial Sequence
  Oligonucleotide
cctcatagacgctatcacatgg

SEQ ID NO: 79 - DNA, 22 bp, Artificial Sequence
  Oligonucleotide
ccatgtgatagcgtctatgagg

SEQ ID NO: 80 - DNA, 21 bp, Artificial Sequence
  Oligonucleotide
ctggcacgacaggtttcccga

SEQ ID NO: 81 - DNA, 47 bp, Artificial Sequence
  Oligonucleotide
agatcctttaattcgagctcggtacccgaagatcttcggcttgaacg

SEQ ID NO: 82 - DNA, 40 bp, Artificial Sequence
  Oligonucleotide
acgtcaaatgcttcacatgtccggtatgttcttggagttc

SEQ ID NO: 83 - DNA, 40 bp, Artificial Sequence
  Oligonucleotide
aacataccggacatgtgaagcatttgacgtcacaataacg

SEQ ID NO: 84 - DNA, 45 bp, Artificial Sequence
  Oligonucleotide
acttaattaaggatccggcgcgccccttgagcacgtgcttgtagg

SEQ ID NO: 85 - DNA, 22 bp, Artificial Sequence
  Oligonucleotide
atgaacaccggcaccttctacc

SEQ ID NO: 86 - DNA, 20 bp, Artificial Sequence
  Oligonucleotide
caggatggagtggctgaacg

SEQ ID NO: 87 - DNA, 20 bp, Artificial Sequence
  Oligonucleotide
gcaaacaaaccaccgctggt